(19) United States
(12) Patent Application Publication
(10) Pub. No.: US 2011/0250137 A1
(43) Pub. Date: Oct. 13, 2011

(54) RADIOISOTOPE-LABELED LYSINE AND ORNITHINE DERIVATIVES, THEIR USE AND PROCESSES FOR THEIR PREPARATION

(75) Inventors: Norman Koglin, Berlin (DE); Lutz Lehmann, Berlin (DE); Holger Siebeneicher, Berlin (DE); Andre Müller, Berlin (DE); Niels Böhnke, Berlin (DE)

(73) Assignee: BAYER SCHERING PHARMA AKTIENGESELLCHAFT, Berlin (DE)

(21) Appl. No.: 13/132,671

(22) PCT Filed: Nov. 26, 2009
(86) PCT No.: PCT/EP2009/008419

§ 371 Date: Jun. 29, 2011

(30) Foreign Application Priority Data
Dec. 4, 2008 (EP) 08075919.4

(51) Int. Cl. A61K 51/04; C07C 227/14; A61K 31/198; C07C 271/22; C07C 269/06; A61K 31/27; C07C 309/66; C07C 303/02; A61K 31/255; C07F 7/02; A61K 31/695; C07C 247/12; A61K 31/655; C07D 209/48; A61K 31/4035; A61P 35/00; A61P 43/00; C12Q 1/02; C07C 229/26

(52) U.S. Cl. 424/1.89; 562/574; 514/564; 560/25; 514/483; 558/50; 514/517; 556/420; 514/63; 552/12; 514/151; 548/479; 514/417; 435/29

(57) ABSTRACT

The invention relates to the compounds suitable for radiolabeling with a chelator free radioisotope and radiolabeled compounds of the general Formula I.

Said compounds are ornithine or lysine derivatives.

The invention relates to compounds suitable for radiolabeling with chelator free radioisotope and radiolabeled compounds of the general Formula I.

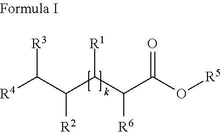

Said compounds are ornithine or lysine derivatives. The invention relates further to the use of said compounds for imaging diseases, methods of preparing such compounds, compositions comprising such compounds, kits comprising such compounds or compositions.

### BACKGROUND

The early diagnosis of malignant tumors plays a very important role in the survival prognosis of a tumor patient. In this diagnosis, non-invasive, diagnostic imaging processes are an important tool. In recent years, PET technology (Positron Emission Tomography), especially, has proven particularly useful. The sensitivity and specificity of PET technology depends significantly on the signal-transmitting substance (tracer) used and its distribution in the body. In the search for suitable tracers, it has been attempted to utilize certain properties of tumors which differentiate tumor tissue from healthy surrounding tissue. Radionuclides used in PET scanning are typically positron emitting isotopes with short half lives such as carbon-11 (˜20 min), nitrogen-13 (˜10 min), oxygen-15 (˜2 min), fluorine-18 (˜110 min), iodine-131 (˜8 days) and iodine-124 (˜4, 2 days). These radionuclides are incorporated either into compounds normally used by the body such as glucose (or glucose analogues), water or ammonia, or into molecules that bind to receptors or other sites of drug action. Such labelled compounds are known as radiotracers. The preferred commercially utilized isotope which is used for PET is ¹⁸F. Owing to its short half life of under 2 hours, ¹⁸F makes particular demands on the preparation of suitable radiotracers. Laborious, long synthesis routes and purifications are not possible with this isotope, since otherwise a considerable part of the radioactivity of the isotope has already decayed before the tracer can be employed for imaging and or diagnosis. It is therefore often not possible to use established synthesis routes for non-radioactive fluorinations in the synthesis of ¹⁸F tracers. In addition, the high specific activity of ¹⁸F (about 80 GBq/nmol) leads to very small amounts of [¹⁸F] fluoride substance for the tracer synthesis, which in turn requires an extreme excess of precursor and makes the success of a radiosynthesis strategy based on non-radioactive fluorination reactions unpredictable.

FDG ([¹⁸F]2-fluorodeoxyglucose)-PET is a widely accepted and widespread tool in the diagnosis and further clinical monitoring of tumors. Malignant tumors compete with the host organism for the glucose supply to the nutrient supply (Warburg O. Über den Stoffwechsel der Carcinomzelle [Concerning the Metabolism of the Carcinoma Cell]. Biochem. Zeitschrift 1924; 152: 309-339; Kellof G. Progress and Promise of FDG-PET Imaging for Cancer Patient Management and Oncologic Drug Development. Clin Cancer Res. 2005; 11(8): 2785-2807). Here, tumor cells usually have an increased glucose metabolism in comparison to surrounding cells of the normal tissue. This tumor specific mechanism is utilized in the use of fluorodeoxyglucose (FDG), a glucose derivative, which is transported into the cells in increased amount, but is metabolically trapped there after phosphorylation as FDG 6-phosphate (“Warburg effect”). ¹⁸F-labeled FDG is therefore an useful tracer for the detection of tumors in patients by means of PET technology. In the search for novel PET tracers, recently amino acids have also increasingly been employed for ¹⁸F PET imaging (e.g. (review): Eur J Nucl Med Mol. Imaging. 2002 May; 29(5):681-90). Here, some of the ¹⁸F-labeled amino acids are suitable for the measurement of the rate of protein synthesis, but most other derivatives for the measurement of direct cell uptake in the tumor. Known ¹⁸F-labeled amino acids are derived, for example, from tyrosine, phenylalanine, proline, asparagine and unnatural amino acids (e.g. J. Nucl Med 1991; 32:1338-1346, J Nucl Med 1996; 37:320-325, J Nucl Med 2001; 42:752-754 and J Nucl Med 1999; 40:331-338).

The present PET tracers which are employed for tumor diagnosis have some indisputable disadvantages: thus although FDG preferably accumulates in those cells having increased glucose metabolism, there is also an increased glucose metabolism in the cells and tissues involved in other pathological and physiological states, for example foci of infection or wound healing (summarized in J. Nucl. Med. Technol. (2005), 33, 145-155). It is often still difficult to decide whether a lesion detected by means of FDG-PET is actually of neoplastic origin or is to be attributed to other physiological or pathological states of the tissue. All in all, diagnostic activity by means of FDG-PET in oncology has a sensitivity of 84% and a specificity of 88% (Gambhir et al. “A tabulated summary of the FDG PET literature” J. Nucl. Med. 2001, 42, 1-93S). Tumors in the brain can only be very poorly demonstrated, for example, owing to the high accumulation of FDG in healthy brain tissue.

The ¹⁸F-labeled amino acid derivatives known hitherto are in some cases suitable for detecting tumors in the brain ((review): Eur J Nucl Med Mol Imaging. 2002 May; 29(5):681-90), however in other tumors they cannot compete with the imaging properties of the “gold standard” [¹⁸F]2-FDG.

Therefore, there is a clear need for radiotracer showing more efficient disease targeting capability. Said radiotracer shall be able to generate trustable and intensive PET images of the patient.

The metabolic accumulation and retention of the so far F-18-labeled amino acids in tumorous tissue is generally lower than for FDG. Moreover, the accessibility of isomerically pure F-18-labeled non-aromatic amino acids is chemically very highly demanding. Ornithine is an amino acid which plays a role in the urea cycle. Ornithine is one of the products of the action of the enzyme arginase on L-arginine, creating urea. Therefore, ornithine is a central part of the urea cycle, which allows for the disposal of excess nitrogen. Ornithine is not an amino acid coded for by DNA, and, in that sense, is not involved in protein synthesis. However, in mammalian non-hepatic tissues, the main use of the urea cycle is in arginine biosynthesis, so as an intermediate in metabolic processes, ornithine is quite important.

Fluorinated ornithine derivatives have been known for a long time and are described in literature, e.g. 4-fluoro-ornithine (Journal of Fluorine Chemistry, volume 7, issue 4, April (1976), p. 397-407):

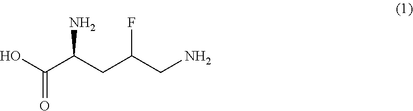

Jandre de Villiers mentions the L-3-fluoro-ornithine derivative (2) whose syntheses however fails. (“Master Thesis”, University of Stellenbosch, 2007, p. 26).

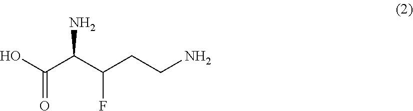

The fluorinated ornithine derivative L-5-(fluoromethyl)-ornithine (3) has been described in literature and in patents (e.g. Tetrahedron: Asymmetry (1997), 8(2), 327-335, WO 9524181 A1, E P326766).

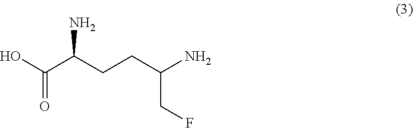

It has been observed that the levels of the enzyme ornithine decarboxylase (ODC) correlate well with the respective tumor stage. The enzyme ODC is the first and rate-limiting enzyme in polyamine biosynthetic pathway. ODC is responsible for intracellular conversion of ornithine into polyamines. A higher protein amount and increased enzymatic activity of ODC was observed for various tumor tissues.

Higher ODC levels and increased enzymatic activity lead to higher polyamines levels in tumor tissues. Polyamine levels itself have been shown to correlate with tumor stage as well, i.e. a higher polyamine content was observed for tumor vs. non-tumor tissue and higher tumor stages showed further increased polyamine content.

Lysine is an amino acid not synthesized in animals and is metabolized in mammals to give acetyl-CoA, via an initial transamination with α-ketoglutarate. The enzymes involved in the initial steps of lysine metabolism are lysine-2-oxoglutarate reductase and saccharopine dehydrogenase (Fellows et al. Biochem J. 1973 October; 136(2): 329-334).

Acetyl-CoA is also an important component in the biogenic synthesis of the neurotransmitter acetylcholine. Choline, in combination with Acetyl-CoA, is catalyzed by the enzyme choline acetyl-transferase to produce acetylcholine and a coenzyme a byproduct.

Also fluorinated lysine derivatives are known: e.g. (5S)-5-fluoro-L-lysine (e.g. Journal of Medicinal Chemistry; 47; 4; (2004); 900-906) or e.g. α-N-Boc-4R-fluoro-L-lysine (e.g. Organic and Biomolecular Chemistry; 1; 20; (2003); 3527-3534).

Polyamines are important molecules governing cell proliferation, survival and apoptosis (review: J. Biochem. 139, 27-33, (2006)). Putricine (1,4-diaminobutane) is a biosynthetic precursor for the biosynthesis of the natural polyamines, like spermine and spermidine. Imaging of the polyamine metabolism in tumors is an ambitious goal. Welch et al. (Int. Jour. Radiat. Appl. Instrum. 1986, Vol 37, No. 7, 607-612) developed a synthesis of [¹⁸F]fluoro-putricine. Unfortunately, [¹⁸F]fluoro-putricine turned out to be unsuited for imaging of tumors or imaging of polyamine metabolism of tumor due to relatively high in-vivo release of free [¹⁸F]fluoride.

The object of the present invention is to find novel based amino acid compounds which are suitable for radiolabeling with chelator free radioisotope for disease imaging such as hyperproliferative diseases. The preferred amino acid being ornithine and lysine.

Patients diagnosed with cancer are staged, or classified, according to the anatomic extent of their tumor. Staging is used to select therapy, to estimate prognosis and to facilitate communication to other clinicians and scientists. Staging in patients with solid tumors consists of determining: (1) the anatomic extension of the primary tumor (T), (2) the presence and location of metastases to regional lymph nodes (N), and (3) the presence and location of metastases to distant organs (M) (Zuluaga et al., 1998). PET is increasingly being used in oncology for cancer staging, response assessment, and radiation treatment planning. Obtained PET images provide an essential piece for radiation therapy planning. Current methods to detect and diagnose regional and distant metastases lack sufficient sensitivity and specificity to optimize therapy. Many patients with undetected micrometastases are surely being under treated, whereas other patients who fall into “high risk” groups are given aggressive systemic therapy without ever confirming whether or not their tumor has spread.

There is an urgent need to develop non-invasive imaging modalities to provide accurate and sensitive information down to the molecular level for hyperproliferative diseases detection, staging, and monitoring of therapy.

Systemic radionuclide therapy is a form of radiotherapy that involves administering the source of the radiation into the patient. With systemic radionuclide therapy the physiology of the disease provides a major contribution to the therapy ultimate resulting in the delivery of the radionuclide to the tumor. By using a radioactive material that will be delivered to the tumor by the patient's own physiologic processes, it is possible to deliver a large dose of radiation to certain tumors with a minimal amount of patient manipulation. Radiotracer consisting of a radionuclide and a targeting agent shall be specifically and efficiently vehiculated to the targeting site avoiding unspecific binding resulting background signal during PET imaging. There is an urgent need to develop radiotracers that specifically bound or accumulate at the targeting site involved in hyperproliferative diseases.

It has been surprisingly found that invention compounds are suitable for imaging. Preferably, invention compounds are suitable for PET, SPECT or Micro-PET imaging or in combination with other imaging conventional method such as Computer Tomography (CT), and magnetic resonance (MR) spectroscopy.

It has been surprisingly found that invention compounds are suitable for treatment of hyperproliferative disease known as radiotherapy or competitive therapy. Radiotherapy occurs by use of the radiation properties of the invention chelator free radiolabelled compounds.

It has been surprisingly found that invention compounds are suitable for staging, monitoring of hyperproliferative disease progression, or monitoring response to therapy directed to hyperproliferative diseases.

### SUMMARY

The object is achieved by the provision according to the invention of chelator free radionuclide-labeled lysine or ornithine derivatives according to the general Formula I, including their diastereomers and enantiomers:
The present invention provides novel compounds of Formula I. If these compounds of Formula I have no chelator free radionuclide preferably ¹⁸F or ¹⁹F but instead contain an appropriate leaving group, they are precursor compounds for the synthesis of chelator free radionuclide preferably ¹⁸F-labelled or ¹⁹F-labelled compounds having Formula I. ¹⁹F-labelled compounds having Formula I are standard reference compounds (as identification tool and for quality check) of the synthesis towards chelator free radionuclide-labelled compounds having Formula I. In the following compounds of Formula I which contain an appropriate leaving group and do not contain chelator free radionuclide or ¹⁹F, are also referred to as “precursor compounds having Formula I”. Moreover, those compounds of Formula I which contain chelator free radionuclide and which do not contain an appropriate leaving group or a moiety which is suited to be converted to an appropriate leaving group are also referred to as “chelator free radionuclide-labelled compounds having Formula I”. Moreover, those compounds of Formula I which contain a moiety which is suited to be converted to an appropriate leaving group being part of a precursor compound of Formula I are also referred to as “starting material having Formula I”. Preferably, chelator free radionuclide is ¹⁸F.
The invention further provides a method for imaging diseases and/or diagnosing diseases, the method comprising introducing into a patient a detectable quantity of a chelator free radionuclide, preferably ¹⁸F-labeled, compound of Formula I.
The invention provides also chelator free radionuclide, preferably ¹⁸F-labelled, or ¹⁹F-labelled compounds having Formula I for use as medicament.
The present invention also provides pharmaceutical compositions comprising compounds preferably chelator free radionuclide-labelled compounds having Formula I, and a pharmaceutically acceptable carrier or diluents.
Another aspect of the invention is directed to the use of compounds of Formula I for the manufacture of medicament, preferably the use of ¹⁸F- or ¹⁹-F-labelled compounds having Formula I.
The invention also provides a method for obtaining chelator free radionuclide-labelled compounds having Formula I from precursor compounds having Formula I.
The invention also provides a method for obtaining ¹⁹F-labelled compounds having Formula I from precursor compounds having Formula I.
The invention also provides a kit for preparing a radiopharmaceutical preparation, said kit comprising a sealed vial comprising
a) compound of Formula I or
b) compound of Formula V and a compound of Formula VI
   or mixture thereof.
The invention also provides a method for obtaining “precursor compounds having Formula I” (wherein the leaving group of the precursor compound having Formula I is attached to a sp²-hybridized carbon atom) from a starting compound having Formula I (wherein the chemical functional group which is converted to the leaving group of a precursor compound having Formula I is also attached to a sp²-hybridized carbon atom).
The invention also provides a method for obtaining “precursor compounds having Formula I” (wherein the leaving group of the precursor compound having Formula I is attached to a sp³ hybridized carbon atom) from a starting compound having Formula I (wherein the chemical functional group which is converted to the leaving group of a precursor compound having Formula I is also attached to a sp³ hybridized carbon atom).
The present invention also provides a kit for imaging diseases. More specifically the compounds of this invention are useful for the imaging of hyperproliferative diseases including but not limited to tumors. The invention, therefore, also relates to the use of imaging compounds for diagnosing these diseases as well as for staging and therapy monitoring.
The present invention also provides compounds of Formula I labelled with radioactive iodine isotopes suited for SPECT imaging (I-123; “iodine SPECT compound”) or PET imaging (I-124; “iodine PET compounds”) or radiotherapy (I-125 and I-131; “iodine therapeutic compounds”) or standard reference compound (I-127; “iodine reference standard compounds”).

### DETAILED DESCRIPTION OF THE INVENTION

In a first aspect, the invention relates to compounds of Formula I

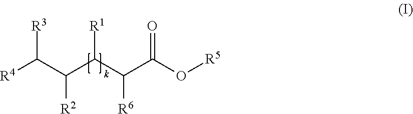

wherein
R¹, R² and R³ are selected independently and individually from the group comprising
a) hydrogen,
b) R⁷—C₁-C₁₀ alkoxy,
c) R⁷—C₁-C₁₀ alkyl,
d) R⁷—C₂-C₁₀ alkenyl,
e) R⁷—C₂-C₁₀ alkinyl,
f) (R⁷-aryl)-C₀-C₁₀alkyl,
g) (R⁷-heteroaryl)-C₀-C₁₀alkyl,
h) ((R⁷—(C₁-C₆)alkoxy)aryl)(C₀-C₁₀)alkyl),
i) R⁷,
j) hydroxyl,
k) C₆-C₁₀ aralkyl,
l) C₁-C₁₀ alkyl and
m) C₁-C₁₀ alkoxy;
   R⁴ is selected from the group comprising
a) NH₂ and
b) R¹⁴;
   R⁵ is selected from the group comprising
a) hydrogen,
b) Z and
c) R¹³;
   R⁶ is selected from the group comprising
a) NH₂ and
b) R¹⁴;
   R⁷ is selected from the group comprising
a) [¹⁹F]fluoro,
b) Chelator free radionuclide,
c) R¹⁵ and
d) R¹⁰;
   R¹⁰ is selected from the group comprising R²⁰ and R³⁰;
   R¹⁵ is a leaving group;
   R¹⁴ is selected from the group comprising
a) N(H)(R⁹),
b) N(R⁹)₂,
c) N═C(R¹¹)(R¹²),
d) 1,3-dioxo-1,3-dihydro-2H-isoindol-2-yl(phthalimido) and
e) azido group;
   R¹³ is a carboxylic acid protecting group;
   R²⁰ is selected from the group comprising
   a) iodo,
   b)—Sn((C₁-C₆)alkyl)₃,
   c)—B(OR⁶⁰)(OR⁶¹) wherein B means boron and
   d)—NMe₂;
   R³⁰ is hydroxyl;
   Z is a metal ion equivalent;
   R⁹ is an amino-protecting group;
   R¹¹ and R¹² are independently and individually selected from the group comprising
a) C₁-C₅ alkyl,
b) substituted or unsubstituted aryl,
c) substituted or unsubstituted aralkyl and
d) substituted or unsubstituted heteroaryl;
   R⁶⁰ and R⁶¹ are independently and individually selected from the group comprising hydrogen, (C₁-C₆)alkyl and cycloalkyl, whereas R⁶⁰ and R⁶¹ can be linked to each other by a single bond or a “methylene bridge”;
   k is an integer from 1 to 4;
   including all isomeric forms of said compound, including but not limited to enantiomers and diastereoisomers as well as racemic mixtures;
   and any pharmaceutically acceptable salt, ester, amide, complex or prodrug thereof.

In one embodiment, the invention is directed to compounds of Formula I

with the proviso that compounds of Formula I contain at least one R⁷. Preferably, compounds of Formula I contain 2 to 3 R⁷. More preferably, compounds of Formula I contain exactly one R⁷.

In one embodiment, the invention is directed to compounds of Formula I wherein R¹, R² and R³ are selected individually and independently from the group comprising
a) hydrogen,
b) R⁷—C₁-C₆ alkoxy,
c) R⁷—C₁-C₆ alkyl,
d) R⁷—C₂-C₆ alkenyl,
e) R⁷—C₂-C₆ alkinyl,
f) (R⁷-aryl)-C₁-C₆alkyl,
g) (R⁷-heteroaryl)-C₁-C₆alkyl,
h) R⁷,
i) hydroxyl and
j) C₁-C₅ alkyl.

Preferably, R¹, R² and R³ are selected individually and independently from the group comprising
a) hydrogen,
b) R⁷—C₁-C₆ alkoxy,
c) R⁷—C₁-C₆ alkyl,
d) R⁷—C₂-C₆ alkenyl,
e) (R⁷-phenyl)-C₁-C₄alkyl,
f) (R⁷-pyridyl)-C₁-C₄alkyl,
g) R⁷ and
h) C₁-C₅ alkyl.

More preferably, R¹, R² and R³ are selected individually and independently from the group comprising
a) hydrogen,
b) R⁷—C₁-C₆ Alkyl,
c) R⁷ and
d) C₁-C₅ Alkyl.

Even more preferably, R¹, R² and R³ are selected individually and independently from the group comprising
a) hydrogen,
b) R⁷—C₁-C₅ alkyl,
c) R⁷ and
d) C₁-C₅ alkyl.

Even more preferably, R¹, R² and R³ are selected individually and independently from the group comprising
a) hydrogen,
b) R⁷—C₁-C₅ alkyl and
c) R⁷.

In one embodiment, the invention is directed to compounds of Formula I wherein R¹, R² and R³ are selected individually and independently from the group comprising
a) hydrogen,
b) R⁷—C₁-C₁₀ alkyl,
c) C₁-C₁₀ alkyl,
d) hydroxyl,
e) C₁-C₁₀ aralkyl and
f) C₁-C₁₀ alkoxy;
   with the proviso that compounds of Formula I contain exactly one R⁷;
   in a preferred embodiment, the invention is directed to compounds of Formula I wherein R¹, R² and R³ are selected individually and independently from the group comprising
a) hydrogen,
b) R⁷—C₂-C₁₀ alkyl,
c) C₁-C₁₀ alkyl,
d) hydroxyl,
e) C₁-C₁₀ aralkyl and
f) C₁-C₁₀ alkoxy.

in a more preferred embodiment, the invention is directed to compounds of Formula I wherein R¹, R² and R³ are selected individually and independently from the group comprising
a) hydrogen,
b) R⁷—C₃-C₁₀ alkyl,
c) C₁-C₁₀ alkyl,
d) hydroxyl,
e) C₁-C₁₀ aralkyl and
f) C₁-C₁₀ alkoxy.

in an even more preferred embodiment, the invention is directed to compounds of Formula I wherein R¹, R² and R³ are selected individually and independently from the group comprising
a) hydrogen,
b) R⁷—C₄-C₁₀ alkyl,
c) C₁-C₁₀ alkyl,
d) hydroxyl,
e) C₁-C₁₀ aralkyl and
f) C₁-C₁₀ alkoxy.

In one embodiment, the invention is directed to compounds of Formula I wherein

R⁷ is selected from the group comprising
a) chelator free radionuclide,
b) R¹⁵ and
c) R¹⁰.

In another embodiment, the invention is directed to compounds of Formula I wherein

R⁷ is selected from the group comprising
a) ¹⁹F,
b) chelator free radionuclide and
c) R¹⁵.

in yet another embodiment, the invention is directed to compounds of Formula I wherein

R⁷ is selected from the group comprising
a) chelator free radionuclide,
b) R¹⁵ and
c) R¹⁰.

in yet another embodiment, the invention is directed to compounds of Formula I wherein

R⁷ is selected from the group comprising
a) chelator free radionuclide and
b) R¹⁵.

in yet another embodiment, the invention is directed to compounds of Formula I wherein
R⁷ is chelator free radionuclide.

Preferably, R⁷ is R¹³ or R¹⁵.

In one embodiment, the invention is directed to compounds of Formula I wherein

R⁷ is [¹⁹F]fluoro.

When R⁷ is [¹⁹F]fluoro, then the present compound can be used as reference compound for in-vitro and in-vivo assay and as medicament (therapeutical agent).

In one embodiment, the invention is directed to compounds of Formula I wherein

R⁷ is a chelator free radionuclide or is comprising a chelator free radionuclide.

Preferably, the chelator free radionuclide is Bromo-77 [⁷⁷Br], Bromo-76 [⁷⁶Br], Oxygen-15 [¹⁵O], Nitrogen-13 [¹³N], Carbon-11 [¹¹C], iodine-123 [¹²³]iodo, iodine-124 [¹²⁴iodo], iodine-125 [¹²⁵iodo], iodine-127 [¹²⁷iodo], iodine-131 [¹³¹iodo] or Fluorine-18 [¹⁸F].

More preferably, the chelator free radionuclide is iodine-123 [¹²³]iodo, iodine-124 [¹²⁴iodo], iodine-125 [¹²⁵iodo], iodine-127 [¹²⁷iodo], or iodine-131 [¹³¹iodo]. Even more preferably, the chelator free radionuclide is iodine-125 [¹²⁵iodo] or iodine-131 [¹³¹iodo] for therapeutical use.

More preferably, when the chelator free radionuclide is Carbon-11 [¹¹C] then R⁷ is ¹¹CH₃, —O(¹¹CH₃), —N(¹¹CH₃)(C₁-C₅)alkyl.

The present invention provides compounds of Formula I labelled with radioactive iodine isotopes suited for SPECT imaging (I-123; “iodine SPECT compound”) or PET imaging (I-124; “iodine PET compounds”) or radiotherapy (I-125 and I-131; “iodine therapeutic compounds”) or standard reference compound (I-127; “iodine reference standard compounds”)

In one embodiment, when R⁷ is selected from the group ¹¹CH₃, —O(¹¹CH₃), —N(¹¹CH₃)(C₁-C₅)alkyl then R⁷ is preferably attached to a sp²-hybridized carbon-atom of Formula I.

In one embodiment, when R⁷ is [¹⁸F[fluoro then R⁴ and R⁶ is NH₂.

More preferably, the chelator free radionuclide is [¹⁸F]fluoro.

When R⁷ is [¹⁸F]fluoro, then the present compound can be used for PET or Micro-PET imaging.

In one embodiment, the invention is directed to compounds of Formula I wherein

### R⁷ is R¹⁰.

In one embodiment, the invention is directed to compounds of Formula I wherein

### R⁷ is R¹⁵.

In one embodiment, the invention is directed to compounds of Formula I wherein

### R⁷ is

a) [¹⁹F]fluoro,
b) [¹⁸F]fluoro,
c) R¹⁵,
d) R¹⁰,
e) [¹²³]iodo,
f) [¹²⁴]iodo,
g) [¹²⁵]iodo,
h) [¹²⁷]iodo and
i) [¹³¹]iodo.

Preferably, R⁷ is selected from the group comprising
a) [¹⁹F]fluoro,
b) [¹⁸F]fluoro,
c) R¹⁵ and
d) R¹⁰.

In one embodiment, the invention is directed to compounds of Formula I wherein when R⁷ is chelator free iodine then R¹, R² and R³ are selected independently and individually from the group comprising
a) hydrogen,
b) (R⁷-aryl)-C₀-C₁₀alkyl,
c) hydroxyl,
d) C₆-C₁₀ aralkyl,
e) C₁-C₁₀ alkyl and
f) C₁-C₁₀ alkoxy.

Preferably, R¹, R² and R³ are selected independently and individually from the group comprising
a) hydrogen and
b) (R⁷-phenyl)-C₁-C₄alkyl.

In one embodiment, the invention is directed to compounds of Formula I wherein R⁴ is NH₂.

In one embodiment, the invention is directed to compounds of Formula I wherein R⁴ is R¹⁴.

In one embodiment, the invention is directed to compounds of Formula I wherein R⁵ is hydrogen.

In one embodiment, the invention is directed to compounds of Formula I wherein R⁵ is R¹³.

In one embodiment, the invention is directed to compounds of Formula I wherein R⁵ is Z. Preferably, Z is selected from the group comprising Na⁺, K⁺, Ca²⁺ and Mg²⁺.

More preferably, Z is Na⁺.

In one embodiment, the invention is directed to compounds of Formula I wherein R⁶ is NH₂.

In one embodiment, the invention is directed to compounds of Formula I wherein R⁶ is R¹⁴.

In one embodiment, the invention is directed to compounds of Formula I wherein R⁹ (amino-protecting group) is selected from the group comprising
a) tert-butoxycarbonyl,
b) allyloxycarbonyl,
c) benzyloxycarbonyl,
d) ethoxycarbonyl,
e) methoxycarbonyl,
f) propoxycarbonyl,
g) 2,2,2-trichlorethoxycarbonyl,
h) 1,1-dimethylpropinyl,
i) 1-methyl-1-phenyl-ethoxycarbonyl,
j) 1-methyl-1-(4-biphenylyl)-ethoxycarbonyl,
k) cyclobutylcarbonyl,
l) 1-methylcyclobutylcarbonyl,
m) vinylcarbonyl,
n) allylcarbonyl,
o) adamantylcarbonyl,
p) diphenylmethylcarbonyl,
q) cinnamylcarbonyl,
r) formyl,
s) benzoyl,
t) trityl,
u) —C(H)(CH₃)C(H)═C(H)—C(O)OR⁵,
v) p-methoxyphenyl-diphenylmethyl and
w) [di-(p-methoxyphenyl)]-phenylmethyl.

Preferably, R⁹ is selected from the group comprising
a) tert-Butoxycarbonyl,
b) formyl,
c) trityl,
d) p-methoxyphenyl-diphenylmethyl and
e) [di-(p-methoxyphenyl)]-phenylmethyl.

More preferably, R⁹ is selected from the group comprising
a) tert-butoxycarbonyl,
b) formyl and
c) trityl.

In one embodiment, R⁹ is tert-butoxycarbonyl;

in another embodiment, R⁹ is formyl;
in yet another embodiment, R⁹ is trityl;

In one embodiment, the invention is directed to compounds of Formula I wherein R¹³ (carboxylic acid protecting group) is selected from the group comprising
a) C₁-C₅ alkyl,
b) C₂-C₅ alkenyl,
c) (C₁-C₅ alkyl-(O—C₁-C₄ alkyl)ₙ-O—)C₁-C₄ alkyl,
d) C₂-C₅ alkinyl,
e) p-methoxybenzyl and
f) triphenylmethyl;
   wherein n is an integer from 0, 1, 2 or 3.

Preferably, R¹³ is selected from the group comprising
a) methyl,
b) ethyl,
c) tert-butyl,
d) p-methoxybenzyl and
e) triphenylmethyl.

In one embodiment, the invention is directed to compounds of Formula I wherein R¹⁵ (leaving group) is R³³ or R³⁴.

Preferably, R¹⁵ is R³³, this embodiment is preferred if R¹⁵ is attached to a sp²-hybridized C-atom.

Preferably, R¹⁵ is R³⁴, this embodiment is preferred if R¹⁵ is attached to a sp³-hybridized C-atom;

R³³ is selected from the group comprising —I⁺(R²⁵)(X⁻), —I⁺(R²⁶)(X⁻), nitro, —N⁺(Me)₃(X⁻), chloro and bromo.

Preferably, R³³ is selected from the group comprising —I⁺(R²⁵)(X⁻), —I⁺(R²⁶)(X⁻), nitro, —N⁺(Me)₃(X⁻), and bromo.

More preferably, R³³ is selected from the group comprising —I⁺(R²⁵)(X⁻), —I⁺(R²⁶)(X⁻), nitro and —N⁺(Me)₃(X⁻).

Even more preferably, R³³ is selected from the group comprising —I⁺(R²⁵)(X⁻) and —I⁺(R²⁶)(X⁻).

Even more preferably, R³³ is nitro.

Even more preferably, R³³ is N⁺(Me)₃(X⁻).

R³⁴ is a leaving group known or obvious to someone skilled in the art and which is taken from but not limited to those described or named in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O-nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15.

R³⁴ is selected from the group comprising chloro, bromo and iodo, mesyloxy, tosyloxy, trifluormethylsulfonyloxy, nona-fluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutyl-phenyl)sulfonyloxy and (4-methoxy-phenyl)sulfonyloxy.

Preferably, R³⁴ is selected from the group comprising iodo, bromo, chloro, mesyloxy, tosyloxy, (4-nitro-phenyl)sulfonyloxy and (2-nitro-phenyl)sulfonyloxy.

More preferably, R³⁴ is selected from the group comprising mesyloxy, tosyloxy and (4-nitro-phenyl)sulfonyloxy.

R²⁵ is substituted or unsubstituted aryl.

Preferably, R²⁵ is selected from the group comprising phenyl, (4-methyl)-phenyl, (4-methoxy)-phenyl, (3-methyl)-phenyl, (3-methoxy)-phenyl, (4-(dimethylcarbamoyl)(methyl)amino)phenyl and naphthyl.

More preferably, R²⁵ is selected from the group comprising phenyl, (4-methyl)-phenyl and (4-methoxy)-phenyl.

Even more preferably, R²⁵ is selected from the group comprising phenyl and (4-methoxy)-phenyl.

More preferably, R²⁵ is (4-(dimethylcarbamoyl)(methyl)amino)phenyl.

R²⁶ is substituted or unsubstituted heteroaryl.

Preferably, R²⁶ is selected from the group comprising 2-furanyl, and 2-thienyl.

More preferably, R²⁶ is 2-thienyl.

X⁻ is selected from the group comprising
a) anion of an inorganic acid and
b) anion of an organic acid.

Preferably, X⁻ is selected from the group comprising
a) CH₃S(O)₂O⁻,
b) ((4-methyl)phenyl)S(O)₂O⁻,
c) CF₃S(O)₂O⁻,
d) C₄F₉S(O)₂O⁻,
e) CF₃C(O)O⁻,
f) H₃CC(O)O⁻,
g) iodide anion,
h) bromide anion,
i) chloride anion,
j) perchlorate anion (ClO₄⁻) and
k) phosphate anion.

More preferably, X⁻ is selected from the group comprising
a) CF₃S(O)₂O⁻
b) C₄F₉S(O)₂O⁻,
c) iodide anion,
d) bromide anion and
e) CF₃C(O)O⁻.

Even more preferably, X⁻ is selected from the group comprising
a) CF₃S(O)₂O⁻,
b) bromide anion and
c) CF₃C(O)O⁻.

In one embodiment, R¹⁰ is preferably R²⁰, if R¹⁵ is attached to a sp²-hybridized C-atom.

In another embodiment, R¹⁰ is preferably R³⁰, if R¹⁵ is attached to a sp^{a}-hybridized C-atom.

In one embodiment, R²⁰ is selected from the group comprising —Sn((C₁-C₆)alkyl)₃, and —B(OR⁶⁰)(OR⁶¹).

In another embodiment, R²⁰ is —NMe₂.

In yet another embodiment, R²⁰ is iodo.

In one embodiment, the invention is directed to compounds of Formula I wherein k is an integer from 1 to 3.

Preferably, k is an integer 1 or 2.

More preferably, k is an integer 1.

More preferably, k is an integer 2.

In one embodiment, the invention is directed to compounds of Formula I wherein n is an integer from 0 to 3.

Preferably, n is an integer 1 or 2.

More preferably, n is an integer 1.

More preferably, n is an integer 2.

R⁶⁰ and R⁶¹ are independently and individually selected from the group comprising hydrogen, (C₁-C₆)alkyl and cycloalkyl, whereas R⁶⁰ and R⁶¹ can be linked to each other by a bond or by a methylene “bridge”.

It is not intended to claim the compound disclosed in WO2009/027727A2 and as reported below

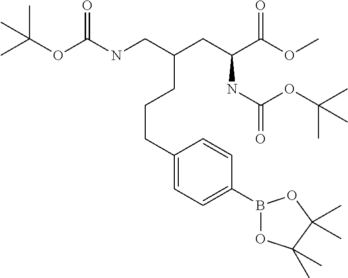

### methyl N²,N⁵-bis(tert-butoxycarbonyl)-4-{3-[4-(4,4,5,5-tetramethyl-1,3,2-dioxaborolan-2-yl)phenyl]propyl}-L-ornithinate

Invention compounds are selected from but not limited to
(4R)—N⁵-[(benzyloxy)carbonyl]-N²-(tert-butoxycarbonyl)-4-hydroxy-L-ornithinate (26)

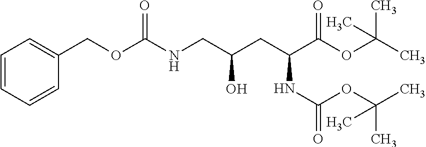

(4R)—N⁵-[(benzyloxy)carbonyl]-N²-(tert-butoxycarbonyl)-4-[(methylsulfonyl)oxy]-L-ornithinate (27)

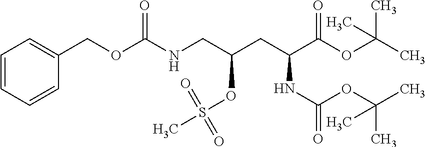

(4R)—N⁵-[(benzyloxy)carbonyl]-N²-(tert-butoxycarbonyl)-4-{[(4-methylphenyl)sulfonyl]oxy}-L-ornithinate (28)

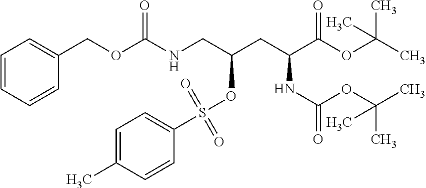

(4S)-[¹⁸F]-fluoro-L-ornithine (29)

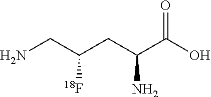

(3R)—N²,N⁵-bis(tert-butoxycarbonyl)-3-fluoro-L-ornithinate (30)

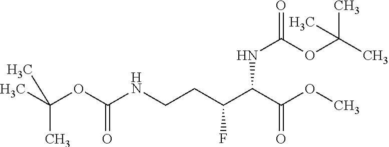

(3R)-3-Fluoro-L-ornithin (31)

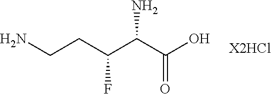

methyl-(5S)—N-(tert-butoxycarbonyl)-6-{[tert-butyl(dimethyl)silyl]oxy}-5-hydroxy-L-norleucinate (34)

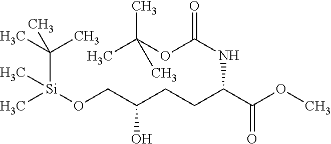

methyl-(5R)-5-azido-N-(tert-butoxycarbonyl)-6-{[tert-butyl(dimethyl)silyl]oxy}-L-norleucinate (35)

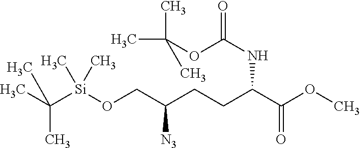

methyl-(5R)-5-azido-N-(tert-butoxycarbonyl)-6-hydroxy-L-norleucinate (36)

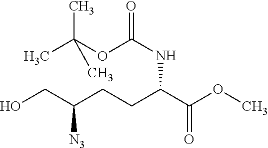

(5R)-[¹⁸F]-fluoromethyl-L-ornithine (38)

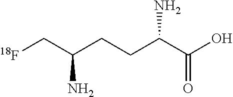

(2S)-5-amino-2-(1,3-dioxo-1,3-dihydro-2H-isoindol-2-yl)-4-fluoropentanoic acid (43)

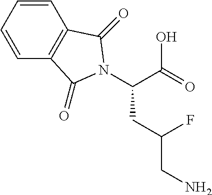

benzyl (2S)-5-azido-2-(1,3-dioxo-1,3-dihydro-2H-isoindol-2-yl)-4-fluoropentanoate (42)

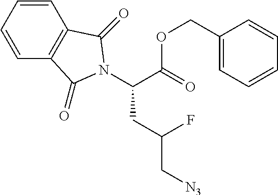

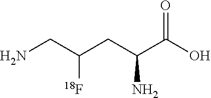

4-(18F)fluoro-L-ornithine

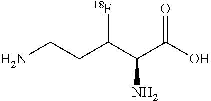

3-(¹⁸F)fluoro-L-ornithine

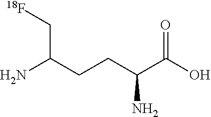

5-amino-6-(¹⁸F)fluoro-L-norleucine

Compounds of Formula I, wherein R⁷ is [¹⁹F]fluoro corresponds to

standard reference compounds. Said compounds are preferably suitable for in-vitro assay, as standard reference in commercialized kit as identification tool and for quality check.

It has been found out that compounds of Formula I, wherein R⁷ is [¹⁸F]fluoro, [¹²³I]iodo, [¹²⁴I]iodo or [¹³¹I]iodo, preferably R⁷ is [¹⁸F]fluoro, do release their radio isotope in-vivo to a relatively small extend (compared to e.g. [¹⁸F]fluoro-putricine) so that tumor imaging or imaging of polyamine metabolism in tumors is possible.

In a second aspect, the invention relates to pharmaceutical composition comprising compounds having Formula I or pharmaceutically acceptable salt of an inorganic or organic acid thereof, a hydrate, a complex, an ester, an amide, a solvate or a prodrug thereof and a pharmaceutical acceptable carrier, diluent, excipient or adjuvant.

In one embodiment, the pharmaceutical compositions comprise a compound of Formula I that is a pharmaceutical acceptable salt, hydrate, complex, ester, amide, solvate or a prodrug thereof.

In one embodiment, the pharmaceutical composition is a pharmaceutical composition comprising compounds having Formula I wherein R⁷ is ¹⁹F or [¹⁸F] or mixture thereof.

In one embodiment, the pharmaceutical composition is a pharmaceutical composition comprising standard reference compounds having Formula I wherein R⁷ is ¹⁹F.

In one embodiment, the pharmaceutical composition is a radiopharmaceutical composition wherein R⁷ is a chelator free radionuclide as defined above. Preferably, the chelator free radionuclide is [¹⁸F], [¹²⁵I], [¹³¹I], [¹²³I], or [¹²⁴I]. More preferably, R⁷ is [¹⁸F].

The compounds having Formula I, Ib or Ic according to the present invention, preferably the chelator free radionuclide labelled compounds according to Formula I, Ib or Ic provided by the invention may be administered intravenously in any suitable pharmaceutically acceptable carrier, e.g. conventional medium such as an aqueous saline medium, or in blood plasma medium. Such medium may also contain conventional pharmaceutical materials such as, for example, pharmaceutically acceptable salts to adjust the osmotic pressure, buffers, preservatives and the like. Among the preferred media are normal saline solution and plasma.

Suitable pharmaceutical acceptable carriers are known to someone skilled in the art. In this regard reference can be made to e.g. Remington's Practice of Pharmacy, 13th ed. and in J. of. Pharmaceutical Science & Technology, Vol. 52, No. 5, September-October, p. 238-311, included herein by reference.

The concentration of the compounds of Formula I, Ib or Ic preferably of the ¹⁸F-labelled compound according to the present invention and the pharmaceutically acceptable carrier, for example, in an aqueous medium, varies with the particular field of use. A sufficient amount is present in the pharmaceutically acceptable carrier when satisfactory visualization of the biological target (e.g. a tumor) is achievable.

In accordance with the invention, the radiolabelled compounds having Formula I, Ib or Ic either as a neutral composition or as a salt with a pharmaceutically acceptable counter-ion are administered in a single unit injectable dose. Any of the common carriers known to those with skill in the art, such as sterile saline solution or plasma, can be utilized after radiolabelling for preparing the injectable solution to image various organs, tumors and the like in accordance with the invention. Generally, the unit dose to be administered for a diagnostic agent has a radioactivity of about 0.1 mCi to about 100 mCi, preferably 1 mCi to 20 mCi. For a radiotherapeutic agent, the radioactivity of the therapeutic unit dose is about 10 mCi to 700 mCi, preferably 50 mCi to 400 mCi. The solution to be injected at unit dosage is from about 0.01 ml to about 30 ml. For imaging purposes after intravenous administration, imaging of the organ or disease location in vivo can take place in a matter of a few minutes. However, imaging can take place, if desired, in hours or even longer, after injecting into patients. In most instances, a sufficient amount of the administered dose will accumulate in the area to be imaged within about 0.1 of an hour to permit the taking of PET or Single Photon Emission Computed Tomography (SPECT) images. Any conventional method of PET or SPECT imaging for imaging purposes or in combination with other imaging conventional method such as Computer Tomography (CT), and magnetic resonance (MR) spectroscopy can be utilized in accordance with this invention.

In a third aspect, the invention relates to compounds having Formula I for use as reference compound, medicament (therapeutical agent) or radiopharmaceutical.

In other word, the invention relates to the use of compounds having Formula I as reference compound, medicament or radiopharmaceutical.

Preferably, the invention relates to [¹⁹F]compound having Formula I (wherein R⁷ is [¹⁹F] as defined above) for the use as reference compound, medicament or radiopharmaceutical. More preferably, the invention relates to [¹⁹F]compound having Formula I (wherein R⁷ is [¹⁹F] as defined above) for the use as reference compound.

Preferably, the invention relates to chelator free radiolabelled compound having Formula I (wherein R⁷ is chelator free radionuclide as defined above) for the use as medicament or radiopharmaceutical. More preferably, R⁷ is defined as above wherein all preferred embodiments are enclosed herein.

More preferably, R⁷ is [18F].

The invention relates also to the use of chelator free radiolabelled compound having Formula I, (wherein R⁷ is chelator free radionuclide as defined above) and of [¹⁹F] compounds having Formula I (wherein R⁷ is [¹⁹F] as defined above) for the manufacture of medicament or radiopharmaceutical for treatment of hyperproliferative diseases.

A hyperproliferative disease includes all diseases and conditions that are associated with any sort of abnormal cell growth or abnormal growth regulation, especially in tumors.

Preferably, the hyperproliferative diseases shall mean cancer developing tumor or metastases. More preferably, tumors are malignant tumors of the gastrointestinal or colorectal tract, carcinoma of the liver, pancreas, kidney, bladder, thyroid gland, prostate, endometrium, ovary, testes, melanomocarcinoma, small-cell and non-small-cell bronchial carcinoma, dysplastic carcinoma of the oral mucosa, invasive oral cancer; breast cancer, including hormone-dependent and hormone-independent breast cancer, squamous epithelial carcinoma, neurological cancers including neuroblastoma, glioma, astrocytoma, osteosarcoma, meningioma; soft-tissue sarcoma; hemangioama and endocrine tumors, including hypophyseal adenoma, chromocytoma, paraganglioma, hematological tumors including lymphoma and leukemias; or metastases of one of the abovementioned tumors.

Even more preferably, tumors are malignant tumors of the gastrointestinal or colorectal tract, carcinoma of the liver, pancreas, kidney, bladder, prostate, ovary, small-cell and non-small-cell bronchial carcinoma, breast cancer, including hormone-dependent and hormone-independent breast cancer, squamous epithelial carcinoma, neurological cancers including neuroblastoma, glioma, astrocytoma, osteosarcoma, meningioma; soft-tissue sarcoma; hemangioama and endocrine tumors, including hypophyseal adenoma, chromocytoma, paraganglioma, hematological tumors including lymphoma or metastases of one of the abovementioned tumors.

Even more preferably, tumors are malignant tumors of the gastrointestinal or colorectal tract, carcinoma of the liver, pancreas, prostate, small-cell and non-small-cell bronchial carcinoma, breast cancer, including hormone-dependent and hormone-independent breast cancer, squamous epithelial carcinoma, neurological cancers including neuroblastoma, glioma, hematological tumors including lymphoma or metastases of one of the abovementioned tumors.

It has been surprisingly found that invention compounds are suitable for radiotherapy or competitive therapy. Radiotherapy occurs by use of the radiation properties of the invention chelator free radiolabelled compounds.

The present invention is also directed to a method of treatment of hyperproliferative diseases, as defined above, comprising the step of administrating into a patient a therapeutically effective amount(s) of a chelator free radiolabelled compound having Formula I (wherein R⁷ is chelator free radionuclide as defined above) or [¹⁹F] compounds having Formula I (wherein R⁷ is [¹⁹F] as defined above) and detecting signal.

Above disclosed preferred embodiments are enclosed herein.

In a fourth aspect, the invention relates to compounds having Formula I for use as imaging agent.

Preferably, the invention relates to chelator free radiolabelled compound having Formula I (wherein R⁷ is chelator free radionuclide as defined above) for the use as imaging agent. More preferably, R⁷ is defined as above wherein all preferred embodiments are enclosed herein.

More preferably, R⁷ is [¹⁸F].

In other word, the invention relates to the use of compounds having Formula I as imaging agent.

Preferably, the invention relates to the use of chelator free radiolabelled compound having Formula I, (wherein R⁷ is chelator free radionuclide as defined above) as imaging agent.

More preferably, R⁷ is defined as above wherein all preferred embodiments are enclosed herein.

More preferably, R⁷ is [¹⁸F].

Preferably, the imaging agent is useful for PET, SPECT or Micro-PET imaging or in combination with other imaging conventional method such as Computer Tomography (CT), and magnetic resonance (MR) spectroscopy. More Preferably, the imaging agent is useful for PET imaging.

Preferably, the imaging agent is suitable for imaging hyperproliferative diseases.

A hyperproliferative disease includes all diseases and conditions that are associated with any sort of abnormal cell growth or abnormal growth regulation, especially in tumors.

Preferably, the hyperproliferative diseases shall mean cancer developing tumor or metastases. More preferably, tumors are malignant tumors of the gastrointestinal or colorectal tract, carcinoma of the liver, pancreas, kidney, bladder, thyroid gland, prostate, endometrium, ovary, testes, melanomocarcinoma, small-cell and non-small-cell bronchial carcinoma, dysplastic carcinoma of the oral mucosa, invasive oral cancer; breast cancer, including hormone-dependent and hormone-independent breast cancer, squamous epithelial carcinoma, neurological cancers including neuroblastoma, glioma, astrocytoma, osteosarcoma, meningioma; soft-tissue sarcoma; hemangioama and endocrine tumors, including hypophyseal adenoma, chromocytoma, paraganglioma, hematological tumors including lymphoma and leukemias; or metastases of one of the abovementioned tumors.

Even more preferably, tumors are malignant tumors of the gastrointestinal or colorectal tract, carcinoma of the liver, pancreas, kidney, bladder, prostate, ovary, small-cell and non-small-cell bronchial carcinoma, breast cancer, including hormone-dependent and hormone-independent breast cancer, squamous epithelial carcinoma, neurological cancers including neuroblastoma, glioma, astrocytoma, osteosarcoma, meningioma; soft-tissue sarcoma; hemangioama and endocrine tumors, including hypophyseal adenoma, chromocytoma, paraganglioma, hematological tumors including lymphoma or metastases of one of the abovementioned tumors.

Even more preferably, tumors are malignant tumors of the gastrointestinal or colorectal tract, carcinoma of the liver, pancreas, prostate, small-cell and non-small-cell bronchial carcinoma, breast cancer, including hormone-dependent and hormone-independent breast cancer, squamous epithelial carcinoma, neurological cancers including neuroblastoma, glioma, hematological tumors including lymphoma or metastases of one of the abovementioned tumors.

The present invention is also directed to a method for imaging hyperproliferative diseases, as defined above, comprising the step of introducing into a patient a detectable quantity of a chelator free radiolabelled compound having Formula I (wherein R⁷ is chelator free radionuclide as defined above). Additionally, radiations are measured or signal is detected and diagnostic can be established.

Above disclosed preferred embodiments are enclosed herein.

In a fifth aspect, the invention relates to a method for obtaining compounds of Formula I or compound of Formula falling under the general Formula I i.e. Ib and Ic, and

wherein R⁷ is a chelator free radionuclide or [¹⁹F].

Surprisingly four methods have been identified for obtaining compounds of Formula I.

In the first method, the invention is directed to a method for obtaining compounds of Formula I wherein R⁷ is a chelator free radionuclide or [¹⁹F] by reacting compounds of Formula I wherein R⁷ is leaving group with a suitable labeling agent.

Optionally the obtained compounds of Formula I wherein R⁷ is a chelator free radionuclide or [¹⁹F] is deprotected at the amine- and/or carboxylic-protecting group. Deprotection occurs by removing of the protecting group R⁵ and R⁹.

In other words, the method for obtaining compounds of Formula I wherein R⁷ is a chelator free radionuclide or [¹⁹F] comprises the steps
Reacting compound of Formula I wherein R⁷ is leaving group with suitable labeling agent, and
optionally deprotecting amine- and/or carboxylic-protecting group.

Suitable labeling agent is defined as a chemical entity comprising a chelator free radionuclide or [¹⁹F] derivative wherein said chemical entity enables the labeling reaction.

Preferably, the compound of Formula I is protected at the functional OH and NH₂ moieties defined in R⁴, R⁵ and R⁶ as defined above.

Preferably, the leaving group is defined as R⁷ being R¹⁵ as defined above,

Preferably, when R⁷ is R¹⁵ as defined above then R⁴ and R⁶ are R¹⁴ as defined above and R⁵ is R¹³ as defined above.

In one embodiment, the invention is directed to a method for obtaining compounds of Formula I wherein R⁷ is a chelator free radionuclide by reacting compounds of Formula I wherein R⁷ is leaving group with a suitable radiolabeling agent.

Optionally the obtained compounds of Formula I wherein R⁷ is a chelator free radionuclide is deprotected at the amine- and/or carboxylic-protecting group. Deprotection occurs by removing of the protecting group R⁵ and R⁹.

In other words, the method for obtaining compounds of Formula I wherein R⁷ is a chelator free radionuclide comprises the steps
Reacting compound of Formula I wherein R⁷ is leaving group with suitable radiolabeling agent, and
optionally deprotecting amine- and/or carboxylic-protecting group.

Preferably, the leaving group is defined as R⁷ being R¹⁵ as defined above,

Preferably, when R⁷ is R¹⁵ as defined above then R⁴ and R⁶ are R¹⁴ as defined above and R⁵ is R¹³ as defined above.

The term “suitable radiolabeling agent” as employed herein refers to reagents causing reaction conditions which are known or obvious to someone skilled in the art and which are chosen from but not limited to: acidic, basic, hydrogenolytical, oxidative, photolytical, preferably acidic cleavage conditions and which are chosen from but not limited to those described in Greene and Wuts, Protecting groups in Organic Synthesis, third edition, page 494-653 and 249-290, respectively.

R⁷ being chelator free radionuclide is defined as above with all already disclosed embodiments. Preferably, R⁷ is [¹⁸F].

R¹⁵ (leaving group) is defined as above with all already disclosed embodiments.

In one embodiment, the invention is directed to a method for obtaining compounds of Formula I wherein R⁷ is [¹⁸F] by reacting compounds of Formula I wherein R⁷ is leaving group with a suitable Fluoro-radiolabeling agent.

Optionally the compounds of Formula I wherein R⁷ is [¹⁸F] is deprotected at the amine- and/or carboxylic-protecting group. Deprotection occurs by removing of the protecting group R⁵ and R⁹.

In other words, the method for obtaining compounds of Formula I wherein R⁷ is [¹⁸F] comprises the step
Reacting compound of Formula I wherein R⁷ is leaving group with suitable Fluoro-radiolabeling agent, and
Optionally deprotecting amine- and/or carboxylic-protecting group.

Preferably, the Fluoro-radiolabeling agent is a compound comprising F-anions (F meaning ¹⁸F) More preferably, F-fluoro-radiolabeling agent is selected from the group comprising 4,7,13,16,21,24-hexaoxa-1,10-diazabicyclo[8.8.8]-hexacosane KF, i.e. crownether salt Kryptofix KF, KF, HF, KHF₂, CsF, NaF and tetraalkylammonium salts of F, such as tetrabutylammonium fluoride, and wherein F═¹⁹F.

Preferably, the leaving group is defined as R⁷ being R¹⁵ as defined above.

R¹⁵ (leaving group) is defined as above with all already disclosed embodiments.

When the compound according to Formula I comprising a leaving group is additionally defined as following R⁷ is R¹⁵ then R⁴ and R⁶ are R¹⁴ are preferably defined as above and R⁵ is R¹³.

When the compound according to Formula I comprising a leaving group is additionally defined as following

### R⁷ is R¹⁵,

### R⁴ is R¹⁴,

### R⁶ is R¹⁴ and

### R⁵ is R¹³

then the obtained fluoro-radiolabeled compounds of Formula I is preferably a compound wherein R⁴ and R⁶ and R⁵ are hydrogen.

The term “radiolabelling” a molecule, as used herein, usually refers to the introduction of a radionuclide such as ¹⁸F-atom into the molecule.

In one embodiment, the invention is directed to a method for obtaining compounds of Formula I wherein R⁷ is [¹⁹F] by reacting compounds of Formula I wherein R⁷ is leaving group with a suitable Fluoro-labeling agent.

Optionally the compounds of Formula I wherein R⁷ is [¹⁸F] is deprotected at the amine- and/or carboxylic-protecting group. Deprotection occurs by removing of the protecting group R⁵ and R⁹.

In other words, the method for obtaining compounds of Formula I wherein R⁷ is [¹⁹F] comprises the step
Reacting compound of Formula I wherein R⁷ is leaving group with suitable Fluoro-labeling agent, and
Optionally deprotecting amine- and/or carboxylic-protecting group.

Preferably, the Fluoro-labeling agent is a compound comprising F-anions (F meaning [¹⁹F]). More preferably, F-fluoro-radiolabeling agent is selected from the group comprising 4, 7, 13, 16, 21,24-hexaoxa-1,10-diazabicyclo[8.8.8]-hexacosane KF, i.e. crownether salt Kryptofix KF, KF, HF, KHF₂, CsF, NaF and tetraalkylammonium salts of F, such as tetrabutylammonium fluoride, and wherein F═[¹⁹F].

Preferably, the leaving group is defined as R⁷ being R¹⁵ as defined above.

R¹⁵ (leaving group) is defined as above with all already disclosed embodiments.

When the compound according to Formula I comprising a leaving group is additionally defined as following R⁷ is R¹⁵ then R⁴ and R⁶ are R¹⁴ are preferably defined as above and R⁵ is R¹³.

When the compound according to Formula I comprising a leaving group is additionally defined as following

### R⁷ is R¹⁵,

### R⁴ is R¹⁴,

### R⁶ is R¹⁴ and

R⁵ is R¹³ then the obtained fluoro-labeled compounds of Formula I is preferably a compound wherein R⁴ and R⁶ and R⁵ are hydrogen.

Above disclosed preferred embodiments are enclosed herein.

In the second method, the invention is directed to a method for obtaining compounds of Formula Ib

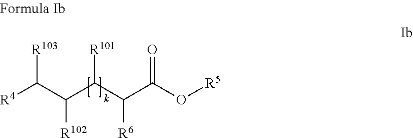

by reacting a compound of Formula V

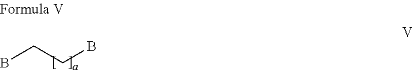

with a labeling agent comprising R⁸⁶ to yield a compound of Formula IV,

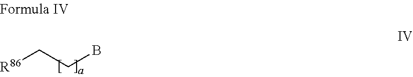

by substituting said compound of Formula IV with a compound of Formula VI

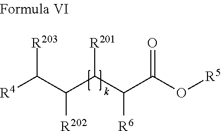

and
Optionally, by deprotecting amine- and/or carboxylic-protecting group;
   wherein Formula Ib is defined as bellowed
   R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
d) C₆-C₁₀ aralkyl,
e) C₁-C₁₀ alkyl and
f) C₁-C₁₀ alkoxy,
   with the proviso that compounds of Formula Ib comprise at least one R⁸⁶,
   R⁸⁶ is a chelator free radionuclide or [¹⁹F], and
   R⁴, R⁵, R⁶, and k are defined as above,
   wherein Formula V is defined as bellowed
   a is an integer from 0 to 5, and
   B is a leaving group,
   wherein Formula IV is defined as bellowed
   a is an integer from 0 to 5,
   B is a leaving group, and
   R⁸⁶ is a chelator free radionuclide or [¹⁹F],
   wherein Formula VI is defined as bellowed
   R²⁰¹, R²⁰² and R²⁰³ are selected individually and independently from the group comprising
a) hydrogen,
b) ((R⁸-aryl)(C₀-C₁₀)alkyl
c) hydroxyl,
d) C₆-C₁₀ aralkyl,
e) C₁-C₁₀ alkyl and
f) C₁-C₁₀ alkoxy;
   R⁸ is hydroxyl;
   with the proviso that compounds of Formula VI comprise at least one R⁸,
   R⁴, R⁵, R⁶, and k are defined as above.

In other words, the method for obtaining compounds of Formula Ib comprises the steps
Reacting compound of Formula V with labelling agent comprising R⁸⁶ to yield a compound of Formula IV,
Substituting compound of Formula IV with a compound of Formula VI, and
Optionally deprotecting amine- and/or carboxylic-protecting group.

### Formula Ib Preferred Embodiments:

Preferably, R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
   with the proviso that compounds of Formula Ib comprise at least one R⁸⁶,

More preferably, one of R¹⁰¹, R¹⁰² and R¹⁰³ is ((R⁸⁶—(C₁-C₆)alkoxy)aryl)(C₀-C₁₀)alkyl).

Preferably, R⁸⁶ is a chelator free radionuclide selected from the group of Bromo-77 [⁷⁷Br], Bromo-76 [⁷⁶Br], Oxygen-15 [¹⁵O], Nitrogen-13 [¹³N], Carbon-11 [¹¹C], iodine-123 [¹²³]iodo, iodine-124 [¹²⁴iodo], iodine-125 [¹²⁵iodo], iodine-127 [¹²⁷iodo], iodine-131 [¹³¹iodo] and Fluorine-18 [¹⁸F].

More preferably, the chelator free radionuclide is iodine-123 [¹²³]iodo, iodine-124 [¹²⁴iodo], iodine-125 [¹²⁵iodo], iodine-127 [¹²⁷iodo], or iodine-131 [¹³¹iodo].

More preferably, the chelator free radionuclide is [¹⁸F] fluoro.

Preferably, R⁸⁶ is [¹⁹F].

Preferably, compounds of Formula Ib comprise 1 or 2 R⁸⁶. More preferably, compounds of Formula Ib comprise exactly one R⁸⁶.

Preferred embodiments disclosed above are included herein for R⁴, R⁵, R⁶, k and chelator free radionuclide.

### Formula V Preferred Embodiments:

Preferably, a is an integer from 0 to 2. More preferably, a is an integer from 0 to 1.

Preferably, B is a leaving group selected individually and independently from the group comprising halo, mesyloxy, tosyloxy, trifluormethylsulfonyloxy, nona-fluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutyl-phenyl)sulfonyloxy and (4-methoxy-phenyl)sulfonyloxy.

More preferably, B is selected from the group comprising iodo, bromo, chloro, mesyloxy, tosyloxy, trifluormethylsulfonyloxy and nona-fluorobutylsulfonyloxy.

Preferably, halo is chloro, bromo or iodo.

### Formula IV Preferred Embodiments:

a and B are defined as for Formula V.

R⁸⁶ is defined as for Formula Ib.

### Formula VI Preferred Embodiments:

Preferably R²⁰¹, R²⁰² and R²⁰³ are selected individually and independently from the group comprising
a) hydrogen,
b) ((R⁸—(C₁-C₆)alkoxy)aryl)(C₀-C₁₀)alkyl)
c) hydroxyl,
   with the proviso that compounds of Formula Ib comprise at least one R⁸,

More preferably, one of R²⁰¹, R²⁰² and R²⁰³ is ((R⁸—(C₁-C₆)alkoxy)aryl)(C₀-C₁₀)alkyl).

R⁸ is hydroxyl.

Preferably, compounds of Formula Ib comprise 1 or 2 R⁸. More preferably, compounds of Formula Ib comprise exactly one R⁸.

Preferred embodiments disclosed above are included herein for R⁴, R⁵, R⁶ and k.

Suitable labeling agent is defined as a chemical entity comprising a chelator free radionuclide or [¹⁹F] derivative wherein said chemical entity enables the labeling reaction.

In a further embodiment, the invention is directed to a method for obtaining compounds of Formula Ib

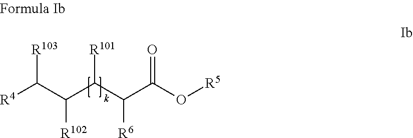

wherein R⁸⁶ is a chelator free radionuclide
by reacting a compound of Formula V

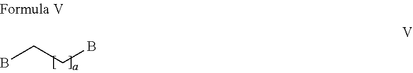

with a suitable radiolabeling agent comprising R⁸⁶ to yield a compound of Formula IV,

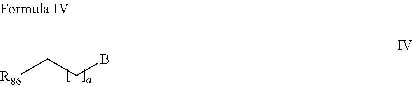

by substituting said compound of Formula IV with a compound of Formula VI

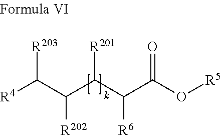

and
optionally, deprotecting amine- and/or carboxylic-protecting group;
wherein Formula Ib is defined as bellowed
R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
d) C₆-C₁₀ aralkyl,
e) C₁-C₁₀ alkyl and
f) C₁-C₁₀ alkoxyl,
   with the proviso that compounds of Formula Ib comprise at least one R⁸⁶,
   R⁸⁶ is chelator free radionuclide, and
   R⁴, R⁵, R⁶, and k are defined as above,
   wherein Formula V is defined as bellowed
   a is an integer from 0 to 5, and
   B is a leaving group,
   wherein Formula IV is defined as bellowed
   a is an integer from 0 to 5,
   B is a leaving group, and
   R⁸⁶ is chelator free radionuclide,
   wherein Formula VI is defined as bellowed
   R²⁰¹, R²⁰² and R²⁰³ are selected individually and independently from the group comprising
a) hydrogen,
b) ((R⁸-aryl)(C₀-C₁₀)alkyl
c) hydroxyl,
d) C₆-C₁₀ aralkyl,
e) C₁-C₁₀ alkyl and
f) C₁-C₁₀ alkoxy;
   R⁸ is hydroxyl;
   with the proviso that compounds of Formula VI comprise at least one R⁸,
   R⁴, R⁵, R⁶, and k are defined as above.

In other words, the method for obtaining compounds of Formula Ib wherein R⁸⁶ is a chelator free radionuclide comprises the steps
Reacting compound of Formula V with suitable radiolabelling agent comprising R⁸⁶ wherein R⁸⁶ is a chelator free radionuclide to yield a compound of Formula IV,
Substituting compound of Formula IV with a compound of Formula VI, and
Optionally deprotecting amine- and/or carboxylic-protecting group.

### Formula Ib Preferred Embodiments:

Preferably, R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
   with the proviso that compounds of Formula Ib comprise at least one R⁸⁶,

More preferably, one of R¹⁰¹, R¹⁰² and R¹⁰³ is ((R⁸⁶—(C₁-C₆)alkoxy)aryl)(C₀-C₁₀)alkyl).

Preferably, R⁸⁶ is a chelator free radionuclide selected from the group of Bromo-77 [⁷⁷Br], Bromo-76 [⁷⁶Br], Oxygen-15 [¹⁵O], Nitrogen-13 [¹³N], Carbon-11 [¹¹C], iodine-123 [¹²³]iodo, iodine-124 [¹²⁴iodo], iodine-125 [¹²⁵iodo], iodine-127 [¹²⁷iodo], iodine-131 [¹³¹iodo] and Fluorine-18 [¹⁸F].

More preferably, the chelator free radionuclide is iodine-123 [¹²³]iodo, iodine-124 [¹²⁴iodo], iodine-125 [¹²⁵iodo], iodine-127 [¹²⁷iodo], or iodine-131 [¹³¹iodo].

More preferably, the chelator free radionuclide is [¹⁸F] fluoro.

Preferably, compounds of Formula Ib comprise 1 or 2 R⁸⁶. More preferably, compounds of Formula Ib comprise exactly one R⁸⁶.

Preferred embodiments disclosed above are included herein for R⁴, R⁵, R⁶, k and chelator free radionuclide.

### Formula V Preferred Embodiments:

Preferably, a is an integer from 0 to 2. More preferably, a is an integer from 0 to 1.

Preferably, leaving group B is known or obvious to someone skilled in the art and which is taken from but not limited to those described or named in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O-nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15.

More preferably, B is a leaving group selected individually and independently from the group comprising halo, mesyloxy, tosyloxy, trifluormethylsulfonyloxy, nona-fluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutyl-phenyl)sulfonyloxy and (4-methoxy-phenyl)sulfonyloxy.

Even more preferably, B is selected from the group comprising iodo, bromo, chloro, mesyloxy, tosyloxy, trifluormethylsulfonyloxy and nona-fluorobutylsulfonyloxy. Preferably, halo is chloro, bromo or iodo.

### Formula IV Preferred Embodiments:

a and B are defined as for Formula V.

R⁸⁶ is defined as for Formula Ib.

### Formula VI Preferred Embodiments:

Preferably R²⁰¹, R²⁰² and R²⁰³ are selected individually and independently from the group comprising
a) hydrogen,
b) ((R⁸—(C₁-C₆)alkoxy)aryl)(C₀-C₁₀)alkyl)
c) hydroxyl,
   with the proviso that compounds of Formula Ib comprise at least one R⁸,

More preferably, one of R²⁰¹, R²⁰² and R²⁰³ is ((R⁸—(C₁-C₆)alkoxy)aryl)(C₀-C₁₀)alkyl). R⁸ is hydroxyl.

Preferably, compounds of Formula Ib comprise 1 or 2 R⁸. More preferably, compounds of Formula Ib comprise exactly one R⁸.

Preferred embodiments disclosed above are included herein for R⁴, R⁵, R⁶ and k.

Suitable radiolabeling agent is defined as a chemical entity comprising a chelator free radionuclide derivative wherein said chemical entity enables the radiolabeling reaction.

In a further embodiment, the invention is directed to a method for obtaining compounds of Formula Ib wherein R⁸⁶ is [¹⁹F] comprises the steps
Reacting compound of Formula V with suitable labelling agent comprising R⁸⁶ wherein R⁸⁶ is [¹⁹F] to yield a compound of Formula IV,
Substituting compound of Formula IV with a compound of Formula VI, and
Optionally deprotecting amine- and/or carboxylic-protecting group.

Suitable Fluoro-labeling agent is a compound comprising F-anions (F meaning [¹⁹F]). More preferably, F-fluoro-radiolabeling agent is selected from the group comprising 4, 7, 13, 16, 21,24-hexaoxa-1,10-diazabicyclo[8.8.8]-hexacosane KF, i.e. crownether salt Kryptofix KF, KF, HF, KHF₂, CsF, NaF and tetraalkylammonium salts of F, such as tetrabutylammonium fluoride, and wherein F═[¹⁹F].

Preferred embodiments disclosed above are included herein.

The term “labeling reagent” as employed herein refers to reagents causing reaction conditions which are known or obvious to someone skilled in the art and which are chosen from but not limited to: acidic, basic, hydrogenolytical, oxidative, photolytical, preferably acidic cleavage conditions and which are chosen from but not limited to those described in Greene and Wuts, Protecting groups in Organic Synthesis, third edition, page 494-653 and 249-290, respectively.

Preferably, radiolabeling agent is a compound consisting of or comprising F-anions. More preferably, the Fluoro-radiolabeling agent is selected from the group comprising 4, 7, 13, 16, 21,24-hexaoxa-1,10-diazabicyclo[8.8.8]-hexacosane KF, i.e. crownether salt Kryptofix KF, KF, HF, KHF₂, CsF, NaF and tetraalkylammonium salts of F, such as tetrabutylammonium fluoride, and wherein F═[¹⁹F].

In a sixth aspect, the invention relates to compounds of Formula Ib, and VI defined below Formula Ib

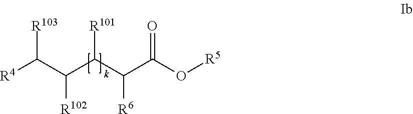

wherein
R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
d) C₆-C₁₀ aralkyl,
e) C₁-C₁₀ alkyl and
f) C₁-C₁₀ alkoxy,
   with the proviso that compounds of Formula Ib comprise at least one R⁸⁶,
   R⁸⁶ is a chelator free radionuclide or [¹⁹F], and
   R⁴, R⁵, R⁶, and k are defined as above,

### Formula Ib Preferred Embodiment:

Preferably R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
   with the proviso that compounds of Formula Ib comprise at least one R⁸⁶,

More preferably, one of R¹⁰¹, R¹⁰² and R¹⁰³ is ((R⁸⁶—(C₁-C₆)alkoxy)aryl)(C₀-C₁₀)alkyl). Preferably, R⁸⁶ is a chelator free radionuclide selected from the group of Bromo-77 [⁷⁷Br], Bromo-76 [⁷⁶Br], Oxygen-15 [¹⁵O], Nitrogen-13 [¹³N], Carbon-11 [¹¹C], iodine-123 [¹²³]iodo, iodine-124 [¹²⁴iodo], iodine-125 [¹²⁵iodo], iodine-127 [¹²⁷iodo], iodine-131 [¹³¹iodo] and Fluorine-18 [¹⁸F].

More preferably, the chelator free radionuclide is iodine-123 [¹²³]iodo, iodine-124 [¹²⁴iodo], iodine-125 [¹²⁵iodo], iodine-127 [¹²⁷iodo], or iodine-131 [¹³¹iodo].

More preferably, the chelator free radionuclide is [¹⁸F]fluoro.

Preferably, R⁸⁶ is [¹⁹F].

Preferably, compounds of Formula Ib comprise 1 or 2 R⁸⁶. More preferably, compounds of Formula Ib comprise exactly one R⁸⁶.

Preferred embodiments disclosed above are included herein for R⁴, R⁵ R⁶ and k.

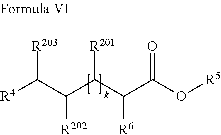

wherein
R²⁰¹, R²⁰² and R²⁰³ are selected individually and independently from the group comprising
a) hydrogen,
b) ((R⁸-aryl)(C₀-C₁₀)alkyl
c) hydroxyl,
d) C₆-C₁₀ aralkyl,
e) C₁-C₁₀ alkyl and
f) C₁-C₁₀ alkoxy;
   R⁸ is hydroxyl;
   with the proviso that compounds of Formula VI comprise at least one R⁸,
   R⁴, R⁵, R⁶, and k are defined as above.

Preferred embodiments disclosed above are included herein.

In a seventh aspect, the invention relates to pharmaceutical composition comprising compounds having Formula Ib or VI mixture thereof or pharmaceutically acceptable salt of an inorganic or organic acid thereof, a hydrate, a complex, an ester, an amide, a solvate or a prodrug thereof and a pharmaceutical acceptable carrier, diluent, excipient or adjuvant.

In one embodiment, the pharmaceutical compositions comprise a compound of Formula Ib, VI or Ic that is a pharmaceutical acceptable salt, hydrate, complex, ester, amide, solvate or a prodrug thereof.

In an eighth aspect, the invention relates to compounds having Formula Ib or VI for use as reference compound, medicament or radiopharmaceutical.

In other word, the invention relates to the use of compounds having Formula Ib or VI as reference compound, medicament or radiopharmaceutical.

The invention relates also to the use of chelator free radiolabelled compound having Formula Ib or VI (wherein R⁸⁶ is chelator free radionuclide as defined above or [¹⁹F]; R⁸ is hydroxyl; R⁴⁰ is chelator free radionuclide as defined above or [¹⁹F] respectively) for the manufacture of a medicament or a radiopharmaceutical for treatment of hyperproliferative diseases.

Preferred embodiments disclosed above relating to the use of compound of Formula I are included herein.

In a ninth aspect, the invention relates to compounds having Formula Ib for use as imaging agent.

In other word, the invention relates to the use compounds having Formula Ib as imaging agent.

The invention relates also to the use of chelator free radiolabelled compound having Formula I, (wherein R⁷ is chelator free radionuclide as defined above) for the manufacture of imaging agent for imaging hyperproliferative diseases.

Preferred embodiments disclosed above relating to the use of compound of Formula I are included herein.

In a tenth aspect, the present invention is directed to a kit comprising a sealed vial comprising a predetermined quantity of a compound

a) compound having Formula I or
b) compound of Formula V and a compound of Formula VI as defined above or mixture thereof.

Preferably, compound having Formula I is a compound wherein R⁷ is R¹⁵ or R¹⁰. The compound will be named precursor for the labelling reaction.

Preferably, compound having Formula I is a compound wherein R⁷ is chelator free radionuclide. The compound will be named radiopharmaceutical that is ready to use for therapy or imaging or that shall undertake deprotecting and/or purification steps before use. Preferably, compound having Formula I is a compound wherein R⁷ is [¹⁹F]. The compound will be named reference compound.

Preferred embodiments disclosed above relating compound of Formula I, V and VI are included herein.

In an eleventh aspect of the present invention is directed to a method for obtaining compounds having
1) Formula I wherein R¹-R⁶ are defined as above, R⁷ is R¹⁵, R⁴ is R¹⁴ and R⁵ is R¹³ as defined above.

In one embodiment the present invention is directed to a method for obtaining precursor compounds having Formula I as defined above wherein R⁷ is R¹⁵, R¹⁵ is R³⁴, R⁴ is R¹⁴, and R⁵ is R¹³ as defined above

comprising the step:
reacting a starting compound of Formula I
   wherein R⁷ is R¹⁰ as defined above, R¹⁰ is R³⁰ as defined above; R⁵ is R¹³ as defined above; and R⁴ is R¹⁴ as defined above;
   with an “electrophilization reagent”.

In one embodiment the present invention is directed to a method for obtaining precursor compounds having Formula I as defined above wherein R⁷ is R¹⁵, R¹⁵ is R³⁴ R⁴ is R¹⁴ and R⁵ is R¹³ as defined above

comprising the step:
reacting a starting compound of Formula I
   wherein R⁷ is R¹⁰ as defined above, R¹⁰ is R³⁰ as defined above; R⁵ is R¹³ as defined above; and R⁴ is R¹⁴ as defined above;
   with an “electrophilization reagent”, with the proviso that R⁷, R¹⁵, R³⁴, R¹⁰ and R³⁰ which are included in compounds having Formula I are attached to a sp^{a} hybridized carbon atom.

In another embodiment the present invention is directed to a method for obtaining precursor compounds having Formula I as defined above wherein R⁷ is R¹⁵, R⁴ is R¹⁴ and R⁵ is R¹³ as defined above, R¹⁵ is R³³ as defined above comprising the step:
reacting a starting compound of Formula I
   wherein R⁷ is R¹⁰, R¹⁰ is R²⁰ as defined above, R⁴ is R¹⁴ and R⁵ is R¹³ as defined above with an “activation reagent”;

In a preferred embodiment the present invention is directed to a method for obtaining precursor compounds having Formula I as defined above wherein R⁷ is R¹⁵, R⁴ is R¹⁴ and R⁵ is R¹³ as defined above, R¹⁵ is R³³ as defined above comprising the step:
reacting a starting compound of Formula I
   wherein R⁷ is R¹⁰, R¹⁰ is R²⁰ as defined above, R⁴ is R¹⁴ and R⁵ is R¹³ as defined above with an activation reagent; with the proviso that R⁷, R¹⁵, R³³, R¹⁰ and R²⁰ which are included in compounds having Formula I are attached to a sp² hybridized carbon atom.

In a twelfth aspect, the present invention is directed to a method for staging, monitoring of hyperproliferative disease progression, or monitoring response to therapy directed to hyperproliferative diseases.

It was surprisingly found that invention compounds of formula I wherein R⁷ is chelator free radionuclide targeting polyamine biosynthetic pathway are taken up to a higher extend in tumor cells than in normal tissues. Thereby, the respective tumor stage will be reflected by radiotracer uptake level.

In one embodiment the method of staging comprises: (i) administering to a mammal an therapeutically effective amount(s) of a compound comprising compounds of formula I wherein R⁷ is chelator free radionuclide, (ii) obtaining an image of the one or more organs or tissues or both of said mammal; (iii) quantifying from said image the involved polyamine biosynthetic pathway which is present in the one or more organs or tissues or both of said mammal, and (iv) utilizing the amount determined and a control amount to arrive at a stage of the pathological condition.

In one embodiment the method of monitoring of hyperproliferative disease progression comprises: (i) administering to a mammal an therapeutically effective amount(s) of a compound comprising compounds of formula I wherein R⁷ is chelator free radionuclide, (ii) obtaining an image of the one or more organs or tissues or both of said mammal; (iii) quantifying from said image the involved polyamine biosynthetic pathway which is present in the one or more organs or tissues or both of said mammal, and (iv) utilizing the amount determined for monitoring of hyperproliferative disease progression.

In one embodiment the method of monitoring a mammal's response to therapy directed to hyperproliferative diseases associated with one or more organs or tissues or both of the mammal comprising (i) administering to a mammal an therapeutically effective amount(s) of a compound comprising compounds of formula I wherein R⁷ is chelator free radionuclide, (ii) obtaining an image of the one or more organs or tissues or both of the mammal, (iii) quantifying from said image the involved polyamine biosynthetic pathway which is present in the one or more organs or tissues or both of the mammal, and (iv) utilizing the amount determined and a control amount to gauge the mammal's response, if any, to a therapy.

Preferably, the method is useful for early monitoring a mammal's response to therapy.

Preferred embodiments disclosed above are included herein.

The present aspect of the invention applies also to compound of formula Ib.

Preferably, invention compounds are L-ornithine derivatives (2S) as herein disclosed.

### DEFINITIONS

The following are some definitions that may be helpful in understanding the description of the present invention. These are intended as general definitions and should in no way limit the scope of the present invention to those terms alone, but are put forth for a better understanding of the following description.

Unless the context requires otherwise or it is specifically stated to the contrary, integers, steps, or elements of the invention recited, herein as singular integers, steps or elements clearly, encompass both singular and plural forms of the recited integers, steps or elements. Throughout this specification, unless the context requires otherwise, the word “comprise”, or variations such as “comprises” or “comprising”, will be understood to imply the inclusion of a stated, step or element or integer or group of steps or elements or integers, but not the exclusion of any, other step or element or integer or group of elements or integers. Thus, in the context of this specification, the term “comprising” means “including principally, but not necessarily solely”. Those skilled in the art will appreciate that the invention described herein is susceptible to, variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.
If chiral centers or other forms of isomeric centers are present in a compound according to the present invention, all forms of such stereoisomers, including enantiomers and diastereoisomers, are intended to be covered herein. Compounds containing chiral centers may be used as racemic mixture or as an enantiomerically enriched mixture or as a diastereomeric mixture or as a diastereomerically enriched mixture, or these isomeric mixtures may be separated using well-known techniques, and an individual stereoisomer maybe used alone. In cases in which compounds have carbon-carbon double bonds, both the (Z)-isomers and (E)-isomers as well as mixtures thereof are within the scope of this invention. In cases wherein compounds may exist in tautomeric forms, such as keto-enol tautomers, each tautomeric form is contemplated as being included within this invention whether existing in equilibrium or predominantly in one form.

In the context of the present invention, preferred suitable salts are pharmaceutically acceptable salts of the compounds according to the invention. The invention also comprises salts which for their part are not suitable for pharmaceutical applications, but which can be used, for example, for isolating or purifying the compounds according to the invention. Pharmaceutically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Pharmaceutically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, dietha-nolamine, triethanolamine, dicyclohexylamine, dimethylaminoethanol, procaine, diben-zylamine, N methylmorpholine, arginine, lysine, ethylenediamine and N methylpiperidine.

As used herein, the term “therapeutically effective amount(s)” includes within its meaning a sufficient but non-toxic amount of a compound or composition of the invention to provide the desired therapeutic or imaging effect. The exact amount required will vary from subject to subject depending on factors such as the species being treated, the age and general condition of the subject, the severity of the condition being treated, the particular compound being administered, the mode of administration and so forth. Thus, it is not possible to specify an exact “therapeutically effective amount”, however for any given case an appropriate “therapeutically effective amount” may be determined by one of ordinary skill in the art using only routine trial and experimentation.

As used herein, the term “treatment” refers to any and all uses which remedy a disease state or symptoms, prevent the establishment of a disease, or otherwise prevent, hinder, retard or reverse the progression of disease or other undesirable symptoms in any way whatsoever.

The term “radionuclide” as employed herein refers to an atom with an unstable nucleus, which is a nucleus characterized by excess energy which is available to be imparted either to a newly-created radiation particle within the nucleus, or else to an atomic electron (see internal conversion). The radionuclide, in this process, undergoes radioactive decay, and emits a gamma ray(s) and/or subatomic particles. These particles constitute ionizing radiation. Radionuclides may occur naturally, but can also be artificially produced.

The term “chelator free radionuclide” as employed herein refers to a radionuclide that is bound covalently and directly to an atom of the targeting molecule and wherein no chelating structure is used for providing a spatial proximity between the radionuclide and the targeting molecule through covalent or non-covalent association. Chelators are chelating structure such as DOTA, DTPA, and EDTA

The chelator free radionuclide are useful for PET, SPECT or Micro-PET or in combination with other imaging conventional method such as Computer Tomography (CT), and magnetic resonance (MR) spectroscopy imaging.

Preferably, chelator free radionuclide is consisting or is comprising a suitable PET or SPECT isotopes of Bromine, Oxygen, Nitrogen, Carbon, Iodine, or Fluorine. More preferably, the suitable PET or SPECT isotopes are Bromo-77 [⁷⁷Br], Bromo-76 [⁷⁶Br], Oxygen-15 [¹⁵O], Nitrogen-13 [¹³N], Carbon-11 [¹¹C], iodine-123 [¹²³]iodo, iodine-124 [¹²⁴iodo], iodine-125 [¹²⁵iodo], iodine-127 [¹²⁷iodo], iodine-131 [¹³¹iodo] or Fluorine-18 [¹⁸F]. More preferably the chelator free radionuclide is Fluorine-18 [¹⁸F].

Chelator free radionuclide comprising Carbon-11 [¹¹C] is preferably, but not limited to, ¹¹CH₃, —O(¹¹CH₃) or —N(¹¹CH₃)(C₁-C₅)alkyl.

The term “targeting molecule” as employed herein refers to ornithine or lysine derivative as disclosed in the present invention.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference.

The term “carboxylic acid protecting group” as employed herein by itself or as part of another group is known or obvious to someone skilled in the art, which is chosen from but not limited to a class of protecting groups described in the textbook Greene and Wuts, Protecting groups in Organic Synthesis, third edition, page 494-653, included herewith by reference namely, methyl, ethyl, tert-butyl, p-methoxybenzyl and triphenylmethyl.

As used hereinafter in the description of the invention and in the claims, the terms “inorganic acid” and “organic acid”, refer to mineral acids, including, but not being limited to: acids such as carbonic, nitric, hydro chloric, hydro bromic, hydro iodic, phosphoric acid, perchloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic, trifluormethansulfonic and sulfanilic acid, respectively.

The term “leaving group” as employed herein by itself or as part of another group is known or obvious to someone skilled in the art, and means that an atom or group of atoms is detachable from a chemical substance by a nucleophilic agent, e.g. fluoride atom. Typically the leaving group is displaced as stable species taking with it the bonding electrons.

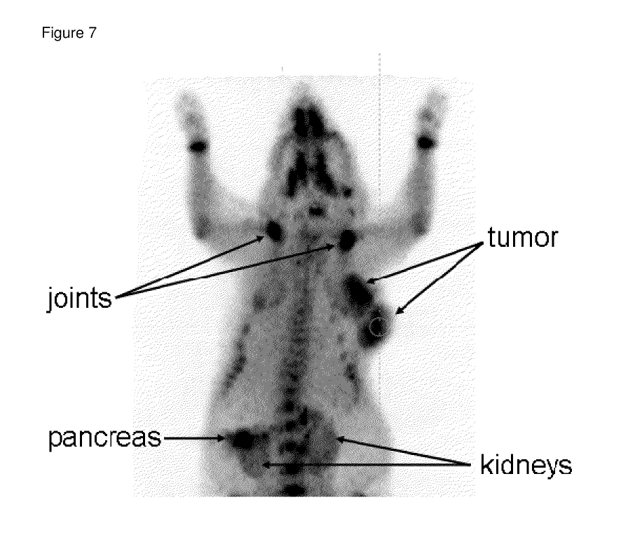

The leaving group is known or obvious to someone skilled in the art and which is taken from but not limited to those described or named in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O— nonaflat”); Carey and Sundberg, Organische Synthese, (1995), page 279-281, table 5.8; Netscher, Recent Res. Dev. Org. Chem., 2003, 7, 71-83, scheme 1, 2, 10 and 15 and others); Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33).

It should be clear that wherever in this description the terms “aryl”, “heteroaryl” or any other term referring to an aromatic system is used, this also includes the possibility that such aromatic system is substituted by one or more appropriate substituents, such as OH, halo, (C₁-C₆)alkyl, CF₃, CN, (C₁-C₆)alkenyl, (C₁-C₆)alkynyl, (C₁-C₆)alkoxy, (dimethylcarbamoyl)(methyl)amino, NH₂, NO₂, SO₃H, —SO₂NH₂, —N(H)C(O)(C₁-C₅)alkyl, —C(O)N(H)(C₁-C₅)alkyl.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl, which themselves can be substituted with one, two or three substituents independently and individually selected from the group comprising halo, nitro, (C₁-C₆)carbonyl, cyano, nitrile, hydroxyl, perfluoro-(C₁-C₁₆)alkyl, in particular trifluormethyl, (C₁-C₆)alkylsulfonyl, (C₁-C₆)alkyl, (C₁-C₆)alkoxy, (dimethylcarbamoyl)(methyl)amino and (C₁-C₆)alkylsulfanyl. As outlined above such “aryl” may additionally be substituted by one or several substituents. It is obvious to someone skilled in the art that afore mentioned substituents can be also combined within one and the same substituents (e.g. halo-alkyl, perfluoroalkyl-alkoxy, ect.) Preferably, aryl is phenyl, naphthyl

The term “heteroaryl” as employed herein refers to groups having 5 to 14 ring atoms; 6, 10 or 14 Π (pi) electrons shared in a cyclic array; and containing carbon atoms (which can be substituted with halo, nitro, ((C₁-C₆)alkyl)carbonyl, cyano, hydroxyl, trifluormethyl, (C₁-C₆)sulfonyl, (C₁-C₆)alkyl, (C₁-C₆)alkenyl, (C₁-C₆)alkynyl, (C₁-C₆)alkoxy or ((C₁-C₆)alkyl)sulfanyl and 1, 2, 3 or 4 oxygen, nitrogen or sulphur heteroatoms (where examples of heteroaryl groups are: thienyl, benzo[b]thienyl, naphtho[2,3-b]thienyl, thianthrenyl, furanyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl and phenoxazinyl groups). Preferably, heteroaryl is pyridyl, 2-furanyl, and 2-thienyl

As outlined above such “heteroaryl” may additionally be substituted by one or several substituents.

The term “Aralkyl” as employed herein refers to a radical in which an aryl group is substituted for an alkyl H atom. Derived from arylated alkyl.

As used hereinafter in the description of the invention and in the claims, the term “alkyl”, by itself or as part of another group, refers to a straight chain or branched chain alkyl group with 1 to 10 carbon atoms such as, for example methyl, ethyl, propyl, isopropyl, butyl, isobutyl, tert-butyl, pentyl, isopentyl, neopentyl, heptyl, hexyl, decyl. Alkyl groups can also be substituted, such as by halogen atoms, hydroxyl groups, C₁-C₄ alkoxy groups or C₆-C₁₂ aryl groups (which, in turn, can also be substituted, such as by 1 to 3 halogen atoms). More preferably alkyl is (C₁-C₁₀)alkyl, (C₁-C₆)alkyl, (C₁-C₅)alkyl, (C₂-C₅)alkyl or (C₁-C₄)alkyl.

As used hereinafter in the description of the invention and in the claims, the term “alkenyl” and “alkynyl” is similarly defined as for alkyl, but contain at least one carbon-carbon double or triple bond, respectively.

As used hereinafter in the description of the invention and in the claims, the term “alkoxy (or alkyloxy)” refer to alkyl groups respectively linked by an oxygen atom, with the alkyl portion being as defined above.

As used herein in the description of the invention and in the claims, the substituent R⁷ as defined above and being attached to the substituents “alkyl”, “alkenyl”, “alkynyl”, “alkoxy” ect. can be attached at any carbon of the corresponding substituent “alkyl”, “alkenyl”, “alkynyl, “alkoxy” ect. Thus, e.g. the term “R⁷—(C₁-C₅)alkoxy” does include different possibilities regarding positional isomerism, e.g. R⁷—(C₅)pentoxy can mean: e.g. R⁷—CH₂—CH₂—CH₂—CH₂—CH₂—O—, CH₃—C(R⁷)H—CH₂—CH₂—CH₂—O— or CH(—CH₂—R⁷)(—CH₃)—CH₂—CH₂—O—, ect.

Whenever the term “substituted” is used, it is meant to indicate that one or more hydrogens attached to the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation to a useful degree of purity from a reaction mixture, and Formulation into a pharmaceutical composition. The substituent groups may be selected from halogen atoms (fluoro, chloro, bromo, iodo), hydroxyl groups, —SO₃H, nitro, (C₁-C₆)alkylcarbonyl, cyano, nitrile, trifluoromethyl, (C₁-C₆)alkylsulfonyl, (C₁-C₆)alkyl, (C₂-C₆)alkenyl, (C₁-C₆)alkynyl, (C₁-C₆)alkoxy and (C₁-C₆)alkylsulfanyl.

The term “halo” or “halogen” refers to fluorine (F), chlorine (Cl), bromine (Br), and iodine (I). If a chiral center or another form of an isomeric center is present in a compound according to the present invention, all forms of such stereoisomer, including enantiomers and diastereoisomers, are intended to be covered herein. Compounds containing a chiral center may be used as racemic mixture or as an enantiomerically enriched mixture or the racemic mixture may be separated using well-known techniques and an individual enantiomer maybe used alone. In cases in which compounds have unsaturated carbon-carbon bonds double bonds, both the (Z)-isomer and (E)-isomers are within the scope of this invention. In cases wherein compounds may exist in tautomeric forms, such as keto-enol tautomers, each tautomeric form is contemplated as being included within this invention whether existing in equilibrium or predominantly in one form.

Unless otherwise specified, when referring to the compounds of Formula the present invention per se as well as to any pharmaceutical composition thereof the present invention includes all of the hydrates, salts, solvates, complexes, and prodrugs of the compounds of the invention. Prodrugs are any covalently bonded compounds, which releases the active parent pharmaceutical according to Formula I.

As used hereinafter in the description of the invention and in the claims, the terms “activation reagent” refers to an “aromatic hypervalent iodo-compound” or an “oxidizing agent” or a “methylation agent”.

As used hereinafter in the description of the invention and in the claims, the terms “methylation agent” refers to chemicals including but not limited to methyl iodide and methyl triflate which are suited to convert an aromatic —NMe₂ group to an aromatic —N⁺Me₃ group (e.g. Chemistry—A European Journal; 13; 8; (2007); 2189-2200; Journal of Fluorine Chemistry; 128; 7; (2007); 806-812).

As used hereinafter in the description of the invention and in the claims, the terms “electrophilization reagent” refers to chemicals including but not limited to carbon tetrabromide (CBr₄), triphenylphosphine/bromine (PPh₃/Br₂), carbon tetrachloride (CCl₄), thionyl chloride (SOCl₂), mesylchloride, mesylanhydride, tosylchloride, tosylanhydride, trifluormethylsulfonylchloride, trifluormethylsulfonylanhydride nona-fluorobutylsulfonylchloride, nona-fluorobutylsulfonylanhydride, (4-bromo-phenyl)sulfonylchloride, (4-bromo-phenyl)sulfonylanhydride, (4-nitro-phenyl)sulfonylchloride, (4-nitro-phenyl)sulfonylchloride, (2-nitro-phenyl)sulfonylchloride, (2-nitro-phenyl)sulfonylanhydride, (4-isopropyl-phenyl)sulfonylchloride, (4-isopropyl-phenyl)sulfonylanhydride, (2,4,6-tri-isopropyl-phenyl)sulfonylchloride, (2,4,6-tri-isopropyl-phenyl)sulfonylanhydride, (2,4,6-trimethyl-phenyl)sulfonylchloride, (2,4,6-trimethyl-phenyl)sulfonylanhydride, (4-tertbutyl-phenyl)sulfonylchloride (4-tertbutyl-phenyl)sulfonylanhydride, (4-methoxy-phenyl)sulfonylchloride, (4-methoxy-phenyl)sulfonylanhydride which are suited to convert an hydroxyl group in a leaving group, thus a sulfonate or halogenide.

As used hereinafter in the description of the invention and in the claims, the term “oxydant” refers to chemicals including but not limited to m-chloroperoxybenzoic, potassium permanganate (KMnO₄), hydrous ruthenium IV oxide (RuO₂xH₂O) with Sodium periodate (NalO₄) and Sodium periodate/ruthenium trichloriode (NalO₄/RuCl₃) which are suited to convert an cyclic sulfamidite to an cyclic sulfamidate (e.g. Tetrahedron 59, (2003), 2581-2616, page 2585 and references cited therein).

As used hereinafter in the description of the invention and in the claims, the term “hyperproliferative diseases” refers to diseases falling under the general wording of cancer (medical term: malignant neoplasm) characterised by uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, do not invade or metastasize. Most cancers form a solid tumor but some, like leukaemia, do not.

As used hereinafter in the description of the invention and in the claims, the term “reference compound” refers to compound differing from the radiotracer in that the reference compound is not radiolabeled as identification tool and for quality check.

As used hereinafter in the description of the invention and in the claims, the term “Micro PET” refers to PET imaging technology designed for high resolution imaging of small laboratory animals.

As used hereinafter in the description of the invention and in the claims, the term “prodrug” means any covalently bonded compound, which releases the active parent pharmaceutical according to Formula I, preferably the ¹⁸F labelled compound of Formula I.

The term “prodrug” as used throughout this text means the pharmacologically acceptable derivatives such as esters, amides and phosphates, such that the resulting in vivo biotransformation product of the derivative is the active drug as defined in the compounds of Formula (I). The reference by Goodman and Gilman (The Pharmaco-logical Basis of Therapeutics, 8 ed, McGraw-HiM, Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxyl group, such as the hydroxyl group on the asymmetric carbon atom, or an amino group is bonded to any group that, when the prodrug is administered to a patient, cleaves to form a free hydroxyl or free amino, respectively. Typical examples of prodrugs are described for instance in WO 99/33795, WO 99/33815, WO 99/33793 and WO 99/33792 all incorporated herein by reference.

Prodrugs can be characterized by excellent aqueous solubility, increased bioavailability and are readily metabolized into the active inhibitors in vivo.

### ABBREVIATIONS AND ACRONYMS

A comprehensive list of the abbreviations used by organic chemists of ordinary skill in the art appears in The ACS Style Guide (third edition) or the Guidelines for Authors for the Journal of Organic Chemistry. The abbreviations contained in said lists, and all abbreviations utilized by organic chemists of ordinary skill in the art are hereby incorporated by reference. For purposes of this invention, the chemical elements are identified in accordance with the Periodic Table of the Elements, CAS version, Handbook of Chemistry and Physics, 67th Ed., 1986-87.

More specifically, when the following abbreviations are used throughout this disclosure, they have the following meanings:
Boc tert-butoxycarbonyl
DAST diethylaminosulfur trifluoride
eq.; equiv. equivalent
h hour, hours
m-CPBA meta-chloroperbenzoic acid
TFA trifluoroacetic acid

### Experimental Data

### Synthesis of [18F]-4-Fluoro-L-ornithine (29)

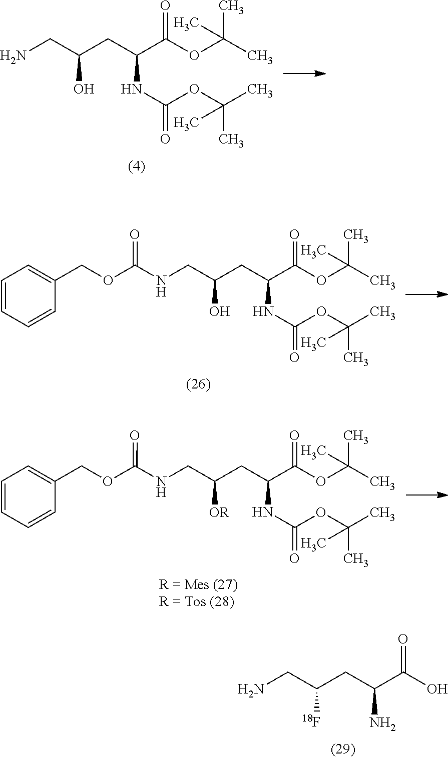

(4) was synthesized according to a procedure described in Org. Lett. 2001, 3, 3153. The free amine functionality was then protected as a benzyloxycarbamate (CbzHN) group which can be cleaved in a hydrogenation step. Besides a Cbz group also other redox-labile amine protecting groups like benzyl or methoyxbenzyl can be employed as well as acid labile protecting groups like triazinones or imide-like moieties like phthloyl groups (P. J. Kocieński, Protecting Groups, 3^{rd} ed, Georg Thieme Verlag 2005, p. 487-591). The free alcohol (26) was converted into a sulphonate capable of reacting with a nucleophilic fluoride ion by reaction with an electrophilizing agent like methanesulphonyl chloride or p-toluenesulphonic acid anhydride, respectively, to give the corresponding precursors (27) and (28). To those skilled of the art also other leaving groups like nosylates, brosylates, nonaflates, triflates, iodides, bromides or chlorides can be employed for the transformation into a precursor (J. March, Advanced Organic Chemistry, 4^{th} ed. 1992, John Wiley & Sons, pp 352ff).

### Synthesis of tert-butyl (4R)—N⁵-[(benzyloxy)carbonyl]-N²-(tert-butoxycarbonyl)-4-hydroxy-L-ornithinate (26)

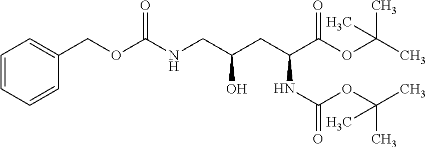

To a solution of dibenzyldicarbonate (600 mg, 2.09 mmol) in tetrahydrofurane (12.5 mL) was added at 0° C. a solution of (4) (455 mg, 1.50 mmol) in tetrahydrofurane/dichloromethane (1:1, 5 mL). After stirring for 1 h at this temperature the reaction was quenched by addition of saturated aqueous sodium bicarbonate solution (20 mL). The layers were separated, the aqueous phase extracted with ethyl acetate (3×20 mL), the combined organic layers dried over sodium sulphate and the solvent removed under reduced pressure. The crude product was purified by column chromatography (silica, hexane/ethyl acetate). Yield: 548 mg, 83%.

¹H-NMR (300 MHz, CHLOROFORM-d): δ [ppm]=1.45 (s, 9H), 1.47 (s, 9H), 1.68-1.84 (m, 1H), 1.90-2.02 (m, 1H), 3.13 (ddd, 1H), 3.28-3.51 (m, 2H), 3.91 (br. s., 1H), 4.22 (dd, 1H), 5.11 (s, 2H), 5.23 (br. s., 1H), 5.39 (br. s., 1H), 7.29-7.40 (m, 5H).

MS (ESIpos): m/z=439 [M+H]⁺

### Synthesis of tert-butyl (4R)—N⁵-[(benzyloxy)carbonyl]-N²-(tert-butoxycarbonyl)-4-[(methylsulfonyl)oxy]-L-ornithinate (27)

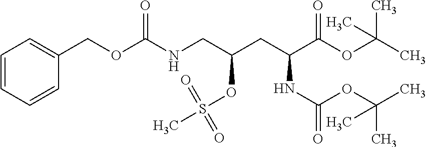

To a solution of (26) (547 mg, 1.25 mmol) in dichloromethane (40 mL) was added at 0° C. triethylamine (0.87 mL, 6.24 mmol) and methanesulphonyl chloride (0.24 mL, 3.12 mmol). After stirring for 3 h at this temperature the reaction mixture was diluted with ethyl acetate (100 mL) and washed with saturated aqueous ammonium chloride solution. The aqueous phase was then extracted twice with ethyl acetate and the combined organic phases were dried over magnesium sulphate. After removal of the solvent the crude product was purified by column chromatography (silica, hexane/ethyl acetate). Yield: 546 mg, 85%.

¹H-NMR (300 MHz, CHLOROFORM-d): δ [ppm]=1.45 (s, 9H), 1.48 (s, 9H), 2.09-2.23 (m, 2H), 3.05 (s, 3H), 3.47-3.67 (m, 2H), 4.27 (br. s., 1H), 4.89 (br. s., 1H), 5.12 (s, 2H), 5.25 (br. s., 2H), 7.29-7.42 (m, 5H).

MS (ESIpos): m/z=517 [M+H]⁺

### Synthesis of tert-butyl (4R)—N⁵-[(benzyloxy)carbonyl]-N²-(tert-butoxycarbonyl)-4-{[(4-methylphenyl)sulfonyl]oxy}-L-ornithinate (28)

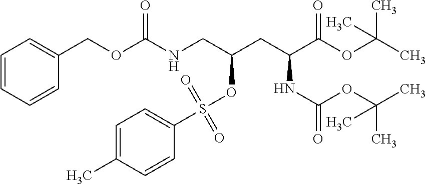

To a solution of (26) (50.0 mg, 0.11 mmol) in dichloromethane/pyridine (4:1, 5 mL) was added at 0° C. p-toluenesulphonic acid anhydride (55.8 mg, 0.17 mmol). After stirring for 2 h at 0° C. and 1 h at room temperature an additional amount of p-toluenesulphonic acid anhydride (55.8 mg, 0.17 mmol) was added. After stirring for 14 h at room temperature the reaction mixture was diluted with ethyl acetate (20 mL) and washed with 2 N hydrochloric acid, brine, and dried over magnesium sulphate. After removal of the solvent the crude product was purified by column chromatography (silica, hexane/ethyl acetate). Yield: 58.2 mg, 68%.

¹H-NMR (400 MHz, CHLOROFORM-d): δ [ppm]=1.43 (s, 9H), 1.45 (s, 9H), 1.92-2.05 (m, 1H), 2.17 (d, 1H), 2.43 (s, 3H), 3.46-3.60 (m, 2H), 4.08-4.24 (m, 1H), 4.73 (br. s., 1H), 5.01-5.15 (m, 4H), 7.29-7.39 (m, 7H), 7.80 (d, 2H).

MS (ESIpos): m/z=593 [M+H]⁺

### Synthesis of (4S)-[¹⁸F]-fluoro-L-ornithine (29)

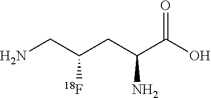

Aqueous [¹⁸F]-fluoride was produced by the ¹⁸O (p,n) ¹⁸F reaction. The [¹⁸F]fluoride (1.64-2.70 GBq) was separated from the target water using a prepared QMA anion exchange column (30 mg, CO3_form) and eluted into a conic glass vial by using 1 mL of a 0.2 M tetrabutylammonium methansulfonate (TBAOMs) in methanol. The solution was dried under a nitrogen flow in the open glass vial at 130° C. To remove residual water, 1.0 mL of acetonitrile was added, and the solution was dried again. This last step was repeated two times and the remaining solid residue was resolubilized in 300 μL acetonitrile containing also 5.0 mg of the precursor tert-butyl (4R)—N-[(benzyloxy)carbonyl]-N-(tert-butoxycarbonyl)-4-[(methylsulfonyl)oxy]-L-ornithinate (27). The glass vial was capped and heated for 15 min at 90° C. After cooling the reaction mixture was diluted with 4 mL acetonitrile/water (1/2 v/v) and subsequently transferred to the HPLC unit using a remote-control-operated HPLC injection system and subjected to a semi-preparative HPLC purification using a Agilent Zorbax Bonus-RP C18, 5 μm; 250_{—}9.4 mm column. Eluent was acetonitrile/water with 0.1 trifluoroacetic acid at a flow of 4 mL/min. For the purification a linear gradient from 40 to 80 acetonitrile within 20 min was used.

The HPLC fraction was diluted with 4 mL water and given on a preconditioned C18 light cartridge. The cartridge was washed with 5 mL water and eluted with 2 mL of ethanol. into a second conic glass vial. 3 mg of palladium on charcoal (Pd/C) (10%) and 4 mg solid ammonium formiate were added to the glass vial and after capping it was heated for 25 min at 90° C. The cooled reaction mixture was passed through a 4 mm HPLC syringe filter into a third conic glass vial to remove the Pd/C. Then 100 μL of 4 N hydrochloric acid were added to the filtrate and the solution was again heated for 15 min at 90° C. in the capped glass vial. The cooled reaction mixture was finally neutralized with 4 N sodium hydroxide (pH 6-8) and sterile filtered to yield 12-31 MBq of the final tracer in a radiochemical yield of 2±1% and a radiochemical purity of 90-99% after a synthesis time of about 153 min.

### Synthesis of (3R)-3-Fluoro-L-ornithin (31)

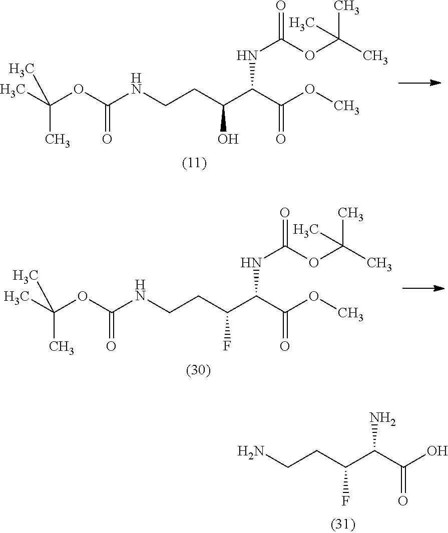

Protected 3-hydroxyornithine (11) was converted into the corresponding 3-fluoro-derivative (30) by reaction with morpholino-sulphurtrifluoride (H. Vorbrüggen, Synthesis 2008, 8, 1165-1174). The deprotection of the protected 3-fluoroornithine was carried out under acidic conditions with hydrochloric acid. To those skilled in the art also other organic or inorganic acids like sulphuric acid or trifluoroacetic acid as well as basic conditions like aqueous sodium hydroxide can be employed for removal of the protecting groups.

### Synthesis of methyl (3R)—N²,N⁵-bis(tert-butoxycarbonyl)-3-fluoro-L-ornithinate (30)

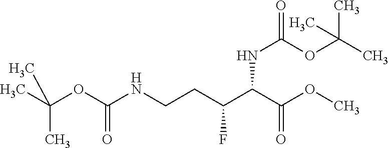

To a solution of alcohol (11) (100 mg, 0.28 mmol) in dichloromethane (10 mL) was added at 0° C. 4-(trifluoro-λ⁴-sulfanyl)morpholine (69 μL, 0.55 mmol) and the mixture was stirred at this temperature for 2 h. Then the reaction mixture was diluted with ethyl acetate (20 mL), washed with saturated aqueous sodium bicarbonate solution, brine, and dried over magnesium sulphate. After removal of the solvent under reduced pressure the residue was purified by preparative HPLC (XBridge C18 5μ 100×30 mm, acetonitrile/1% aqueous formic acid gradient, 50 mL/min) to give the title compound. Yield: 2.4 mg, 3%.

¹H-NMR (400 MHz, CHLOROFORM-d): δ [ppm]=1.45 (s, 9H), 1.47 (s, 9H), 1.71-2.06 (m, 2H), 3.21-3.38 (m, 2H), 3.81 (s, 3H), 4.53 (dd, 1H), 4.72 (br. s., 1H), 5.12 (dd, 1H), 5.22 (d, 1H).

MS (ESIpos): m/z=365 [M+H]⁺

### Synthesis of (3R)-3-fluoro-L-ornithine-dihydrochloride (31)

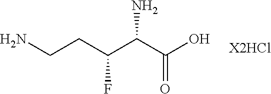

A solution of (30) (2.0 mg, 2.7 μmol) in 6 N hydrochlorid acid was stirred at 80° C. for 3.5 h. After that the mixture was concentrated under reduced pressure, diluted with water an lyophilized to give the title compound as a off-white solid. Yield: 1.0 mg, 82%.

MS (ESIpos): m/z=151 [M−2 HCl+H]⁺

### Synthesis of (5R)-[¹⁸F]-fluoromethyl-L-ornithine (38)

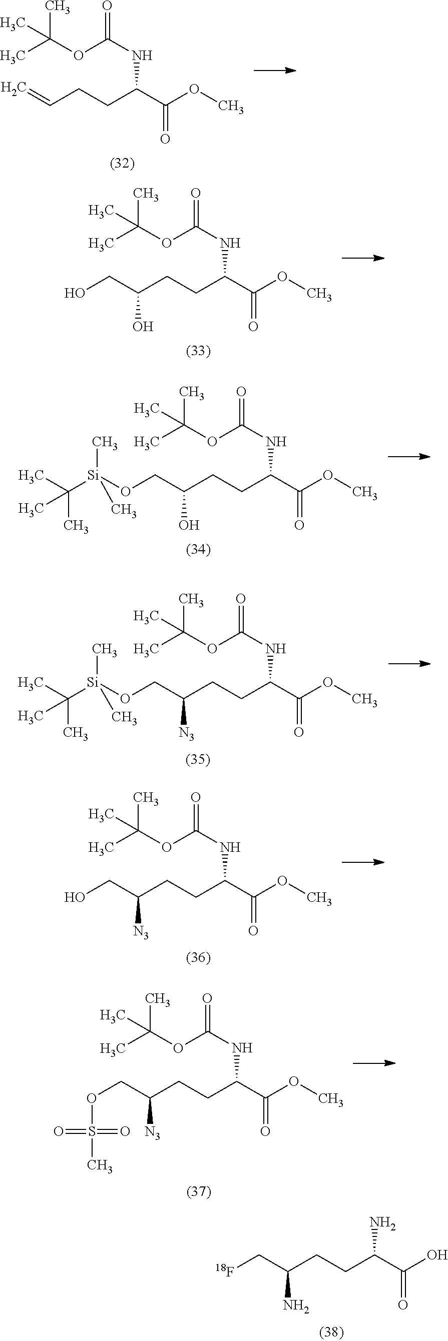

(32) was synthesized according to a procedure described in Org. Biomol. Chem. 2003, 973. The double bond of the homoallylglycine (32) was then asymmetrically dihydroxylated with commercially available AD-mix-α. Apart from that other dihydroxylation procedures known to those skilled in the art like OsO₄ in combination with cooxidants like tert-butyl hydroperoxide (J. Am. Chem. Soc. 1976, 98, 1986), N-methylmorpholino N-oxide (Tetrahedron Lett. 1976, 1973) and others can be employed. Moreover tandem epoxidation-hydrolysis processes according to Jacobsen (Catalytic Asymmetric Synthesis, Ojima, I. Ed., VCH Publishers, 1993, pp 159-202) or Sharpless (Comp. Org. Syn. 1991, 7, 389) can also lead to the desired constitution pattern. The primary alcohol was then chemoselectively protected as a tert-butyl-dimethylsilyl ether and the secondary one was converted to the corresponding azide under Mitsunobu conditions with diphenyl phosphorazidate (DPPA), diethyl azodicarboxylate (DEAD) and PPh₃ (Chem. Eur. J. 2007, 13, 10225). Besides that, a differentiation of the two hydroxyl groups can be achieved by other bulky protecting groups (Protecting Groups, Kocienski P. J., Thieme, 2005, pp 187-364) like pivalate (Tetrahedron 2009, 2226), trityl (Tetrahedron Asymmetry 2009, 78) and others. After deprotection of the primary alcohol (36) the hydroxyl group was converted into the methansulfonate that allows later labeling of the compound with fluorine-18. To those skilled in the art other leaving groups like other sulfonates and halogenides (J. Lab. Cmpd. Rad. 2005, 771) can also act as electrophiles in nucleophilic fluorination reactions.

### Synthesis of methyl-(5S)—N-(tert-butoxycarbonyl)-5,6-dihydroxy-L-norleucinate (33)

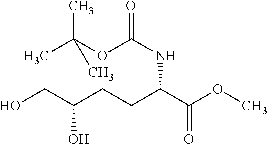

AD-Mix alpha (9.00 g, 1.54 g/mmol) was added to a solution of the alkene (32) (1.42 g, 5.85 mmol) in tert butanol/water (1:1, 40 mL) at 0° C. The suspension was stirred overnight. A saturated solution of aqueous sodium thiosulphate and ethyl acetate were added and the reaction mixture was stirred for 1 h at 25° C. The layers were separated and the aqueous layer was extracted with ethyl acetate (3×). The combined organic layers were washed with saturated aqueous ammonium chloride solution and dried with sodium sulfate. After evaporation the crude product was obtained (758 mg) that was used without further purification in the next step. The diastereomeric excess of (×10) of 78% d.e. was checked by analytic chiral HPLC (Chiralpak AD-H 5 μm 150×4.6 mm, hexane/ethanol 80:20, 1.0 mL/min, 25° C., detection: Corona CAD).

MS (ESIpos): m/z=278 [M+H]⁺

### Synthesis of methyl-(5S)—N-(tert-butoxycarbonyl)-6-{[tert-butyl(dimethyl)silyl]oxy}-5-hydroxy-L-norleucinate (34)

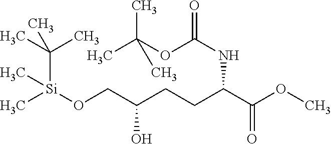

The mixture of diastereomeric diols (33) (758 mg, 2.73 mmol) was dissolved in dichloromethane (40 mL), tert-butyldimethyl silyl chloride (412 mg, 2.73 mmol) and imidazole (279 mg, 4.10 mmol) were added and the mixture was stirred overnight at 25° C. After addition of dichloromethane and water, the layers were separated and the aqueous layer was extracted with dichloromethane (3×). The combined organic layers were dried over sodium sulfate and evaporated to dryness. The residue was purified by flash chromatography n-hexane/ethyl acetate (2:1) to get the desired compound as a colourless oil (518 mg, 1.32 mmol, 48%).

¹³C NMR (75 MHz, CHLOROFORM-d) δ=−5.37 (CH3), 18.26 (C), 25.86 (CH3) (3×), 28.30 (CH3) (3×), 28.40 (CH2), 28.91 (CH2), 52.28 (CH3), 53.21 (CH), 67.02 (CH2), 71.05 (CH), 79.83 (C), 155.41 (C), 173.25 (C) ppm.

MS (ESIpos): m/z=392 [M+H]⁺

### Synthesis of methyl-(5R)-5-azido-N-(tert-butoxycarbonyl)-6-{[tert-butyl(dimethyl)silyl]oxy}-L-norleucinate (35)

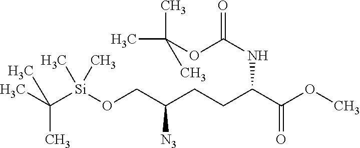

Triphenylphosphine (700 mg, 2.64 mmol), diethyl azodicarboxylate (417 μL, 2.65 mmol), and diphenyl phosphorazidate (342 μL, 1.59 mmol) were added successively to a stirred solution of the alcohol (34) (518 mg, 1.32 mmol) in tetrahydrofurane (10 mL) at room temperature under argon. The reaction mixture was stirred at the same temperature for 3 h. After removal of the solvent in vacuo, the residue was purified by column chromatography using hexane/ethyl acetate (8:1) as the eluent to give the desired compound (439 mg, 1.05 mmol, 80%) as a colorless oil.

¹³C NMR (75 MHz, CHLOROFORM-d) δ=−5.57 (CH3) (2×), 18.19 (C), 25.76 (CH3) (3×), 26.32 (CH2), 28.28 (CH3) (3×), 29.52 (CH2), 52.39 (CH3), 53.18 (CH), 63.17 (CH), 66.17 (CH2), 80.04 (C), 155.26 (C), 172.90 (C) ppm.

MS (ESIpos): m/z=417 [M+H]⁺

### Synthesis of methyl-(5R)-5-azido-N-(tert-butoxycarbonyl)-6-hydroxy-L-norleucinate (36)

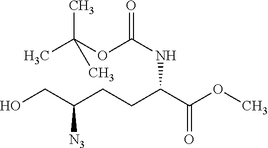

TBDMS-protected azide (35) (439 mg, 1.05 mmol) was dissolved in tetrahydrofurane (30 mL) at 0° C. Then, a 1 M solution of tetrabutyl ammonium fluoride (TBAF, 1.27 mL, 1.27 mmol) in tetrahydrofurane was added to this solution. The mixture was stirred at 25° C. for 1 h. The mixture was concentrated and the residue was subjected to chromatography on silica gel (hexane/ethyl acetate 1:1) to get the desired compound as a colourless oil (292 mg, 0.97 mmol, 92%).

¹³C NMR (75 MHz, CHLOROFORM-d) δ=26.43 (CH2), 28.28 (CH3) (3×), 29.32 (CH2), 52.48 (CH3), 53.18 (CH), 63.62 (CH), 65.20 (CH2), 80.18 (C), 155.34 (C), 172.78 (C) ppm.

MS (ESIpos): m/z=303 [M+H]⁺

### Synthesis of methyl-(5R)-5-azido-N-(tert-butoxycarbonyl)-6-[(methylsulfonyl)oxy]-norleucinate (37)

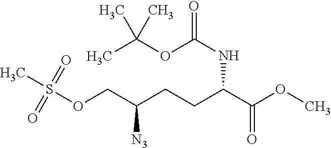

The azidoalcohol (36) (80 mg, 0.27 mmol) and triethylamine (55 μL, 0.40 mmol) were dissolved in dichloromethane (5 mL) and cooled to 0° C. A solution of methanesulphuryl chloride (20 μL, 0.27 mmol) was added slowly. The reaction was gradually warmed to room temperature and stirred for additional 5 h. The reaction mixture was diluted with water and washed with dichloromethane four times, to give a crude product, which was purified by column chromatography (SiO₂:hexanes/ethyl acetate 1:1) to get the desired compound as a colorless oil (110 mg, 0.29 mmol, 99%).

¹³C NMR (151 MHz, CHLOROFORM-d) δ=26.40 (CH2), 28.26 (CH3) (3×), 29.18 (CH2), 37.70 (CH3), 52.61 (CH3), 52.86 (CH), 60.17 (CH), 70.21 (CH2), 80.26 (C), 155.31 (C), 172.54 (C) ppm.

MS (ESIpos): m/z=381 [M+H]⁺

### Synthesis of (5R)-[¹⁸F]-fluoromethyl-L-ornithine (38)

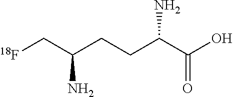

Aqueous [¹⁸F]-fluoride was produced by the ¹⁸O (p,n) ¹⁸F reaction. The [¹⁸F]fluoride (1.51-3.69 GBq) was separated from the target water using a prepared QMA anion exchange column (30 mg, CO₃— form) and eluted into a conic glass vial by using 2 mL of a freshly prepared tetrabutylammonium hydrogencarbonate (TBAHCO3) solution, that was produced by gassing carbon dioxide for 30 min through a solution of 40% tetrabutylammonium hydroxide (5 μL) in acetonitrile/water (9/1 v/v) (2 mL). The solution was dried under a nitrogen flow in the open glass vial at 130° C. To remove residual water, 1.0 ml of acetonitrile was added, and the solution was dried again. This last step was repeated two times and the remaining solid residue was resolubilized in 150 μL 2-methyl-2-butanol containing also 3.0 mg of the precursor methyl-(5R)-5-azido-N-(tert-butoxycarbonyl)-6-[(methylsulfonyl)oxy]-L-norleucinate (37). The glass vial was capped and heated for 30 min at 120° C. After cooling the reaction mixture was diluted with 10 ml acetonitrile/water (9.5/0.5 v/v) and given on a preconditioned C18 Plus cartridge and washed with 30 ml water. The activity was eluted from the cartridge with 1.2 mL acetonitrile into a second conic glass vial and 500 μL 2 N sodium hydroxide were added. The glass vial was heated for 10 min at 80° C. without capping of the vial. After cooling the reaction mixture was diluted with 9 mL water and given on a preconditioned C18 Plus cartridge and washed with 5 mL water for 2 times. The activity was eluted from the cartridge with 1.5 mL acetonitrile into a third conic glass vial and evaporated at 130° C. in the open vial under gentle flow of nitrogen. To remove residual water, 1.0 ml of acetonitrile was added, and the solution was dried again. This last step was repeated once and the solid residue was resolubilized in 500 μL of ethanol. After adding 3 mg of palladium on charcoal (Pd/C) (10%) and 4 mg solid ammonium formiate the capped glass vial was heated for 30 min at 70° C. The cooled reaction mixture was passed through a 4 mm HPLC syringe filter into a fourth conic glass vial to remove the Pd/C. Then 500 μl of 4 N hydrochloric acid were added to the filtrate and the solution was again heated for 5 min at 80° C. in the capped glass vial. The cooled reaction mixture was finally neutralized with 4 N sodium hydroxide (pH 6-8) and sterile filtered to yield 73-97 MBq of the final tracer in a radiochemical yield of 14±7% and a radiochemical purity of 92-97% after a synthesis time of about 210 min.

### Synthesis of 4-fluoro-L-ornithine (2)

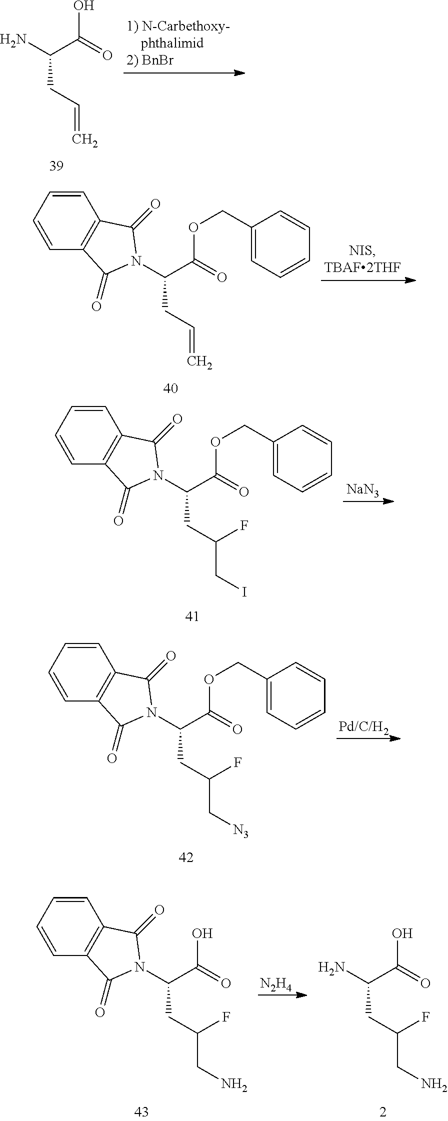

The synthesis of 4-fluoro-L-ornithine (2) was accomplished by iodofluorination of protected (S)-allylglycine (40) (e.g. M. Kuroboshi et al., Tetrahedron Lett. 1991, 32(9), 1215). Also other halofluorination reactions leading to a secondary fluorine functionality like bromofluorination can be used in this step (e.g. J. B. Hester et al., J. Med. Chem. 2001, 44(7), 1099). The terminal iodo group was then substituted by an azide as an nitrogen nucleophile. Besides that, also other nitrogen nucleohiles like phthalimide or benzyl amine can be used in this transformation. Reduction of the azido group and deprotection of the alpha-amine functionality and the carboxy group gave 4-fluoro-L-ornithine (2).

### Synthesis of benzyl (2S)-2-(1,3-dioxo-1,3-dihydro-2H-isoindol-2-yl)pent-4-enoate (40)

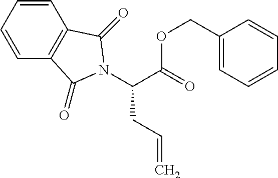

A solution of (S)-allylglycine (100 mg, 0.87 mmol), N-carbethoxyphthalimide (200 mg, 0.91 mmol) and triethylamine (0.17 mL, 1.22 mmol) in dry tetrahydrofurane (10 mL) was stirred under reflux for 14 After drying over sodium sulphate the solvent was removed under reduced pressure.

The residue was dissolved in acetone (3 mL) and potassium carbonate (532 mg, 3.85 mmol) and benzyl bromide (0.17 mL, 1.41 mmol) were added. The mixture was heated to 60° C. for 2 h under microwave irradiation, cooled to. and filtered over Celite. The solvent was removed under reduced pressure and the crude product purified by column chromatography (SiO₂, hexane/ethyl acetate gradient) to give the title compound. Yield: 151 mg, 70%.

¹H-NMR (300 MHz, CHLOROFORM-d): δ [ppm]=2.96-3.11 (m, 2H), 4.96-5.25 (m, 5H), 5.64-5.78 (m, 1H), 7.24-7.37 (m, 5H), 7.70-7.88 (m, 4H).

MS (ESIpos): m/z=336 [M+H]⁺.

### Synthesis of benzyl (2S)-2-(1,3-dioxo-1,3-dihydro-2H-isoindol-2-yl)-4-fluoro-5-iodo-pentanoate (41)

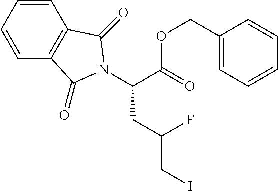

To a solution of (40) (500 mg, 1.49 mmol) and tetra-N-butylammonium dihydrogentrifluorid (2.33 mL, 7.46 mmol) in dry dichloromethane (10 mL) was added at room temperature N-iodosuccinimide (671 mg, 2.98 mmol) portionwise over 2 h and the reaction mixture stirred for further 20 h at this temperature. Then the mixture was diluted with ethyl acetate, washed with saturated sodium bicarbonate solution, brine, and dried over magnesium sulphate. After removal of the solvent the crude product was purified by column chromatography (SiO₂, hexane/ethyl acetate gradient) to give the title compound. Yield: 126 mg, 18%.

¹H-NMR (300 MHz, CHLOROFORM-d): δ [ppm]=2.45-3.08 (m, 2H), 3.31 (dd, 2H), 4.31-4.73 (m, 1H), 5.07-5.30 (m, 3H), 7.20-7.39 (m, 5H), 7.72-7.91 (m, 4H).

¹⁹F-NMR (376 MHz, CHLOROFORM-d): δ [ppm]=−173.46 (m).

MS (ESIpos): m/z=482 [M+H]⁺.

### Synthesis of benzyl (2S)-5-azido-2-(1,3-dioxo-1,3-dihydro-2H-isoindol-2-yl)-4-fluoropentanoate (42)

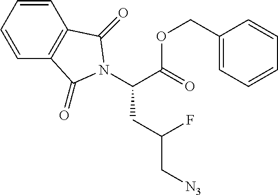

A solution of (41) (110 mg, 0.23 mmol) and sodium azide (74.3 mg, 1.14 mmol) in dry dimethylformamide (10 mL) was heated to 80° C. for 3 h under microwave irradiation. After cooling to room temperature the reaction mixture was diluted with ethyl acetate, washed with saturated sodium bicarbonate solution and brine and concentrated under reduced pressure. The crude product was purified by column chromatography (SiO₂, hexane/ethyl acetate gradient) to give the title compound. Yield: 73 mg, 81%.

¹H-NMR (300 MHz, CHLOROFORM-d): δ [ppm]=2.39-2.75 (m, 2H), 3.34-3.50 (m, 2H), 4.40-4.72 (m, 1H), 5.03-5.25 (m, 3H), 7.18-7.41 (m, 5H), 7.69-7.95 (m, 4H).

MS (ESIpos): m/z=397 [M+H]⁺.

### Synthesis of (2S)-5-amino-2-(1,3-dioxo-1,3-dihydro-2H-isoindol-2-yl)-4-fluoro-pentanoic acid (43)

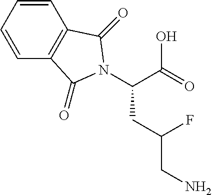

A mixture of (42) (110 mg, 0.278 mmol) and palladium (33 mg, 10% on charcoal, 31 μmol) in methanol (5 mL) was stirred under an hydrogen atmosphere for 3 h at room temperature. Then the mixture was filtrated through Celite, the filter cake washed additional methanol, and the filtrate was concentrated under reduced pressure. Yield: 35.0 mg, 45%.

¹H-NMR (400 MHz, DEUTERIUM OXIDE): δ [ppm]=2.25-2.62 (m, 2H), 3.13-3.41 (m, 2H), 4.78-4.87 (m, 1H), 4.91-5.14 (m, 1H), 7.76-7.89 (m, 4H).

¹⁹F-NMR (376 MHz, DEUTERIUM OXIDE): δ [ppm]=−191.17 (m, 0.5 F), −191.71 (m, 0.5 F).

MS (ESIpos): m/z=281 [M+H]⁺.

### Bio-Data

### Example (38) (5R)[¹⁸F]-fluoromethyl-L-ornithine

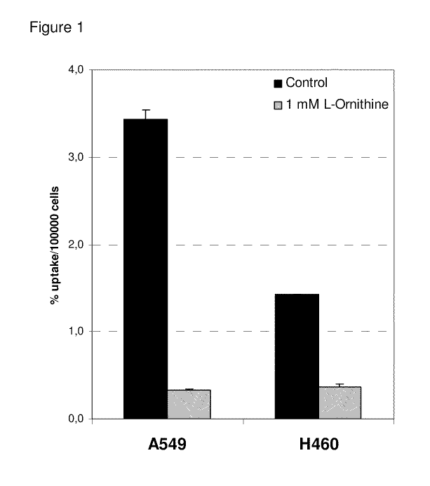

To determine the specificity of (5R)-[¹⁸F]-fluoromethyl-L-ornithine (38), the fluorinated compound was used as tracer in a cell competition experiment in A549 (human NSCLC) as well as H460 (human NSCLC) tumor cells using an excess of L-ornithine (1 mM) for competition. Surprisingly, it was discovered, that the uptake of (5R)-[¹⁸F]-fluoromethyl-L-ornithine was blockable by excess of L-ornithine, indicating the use of the same transport system for uptake (FIG. 1).

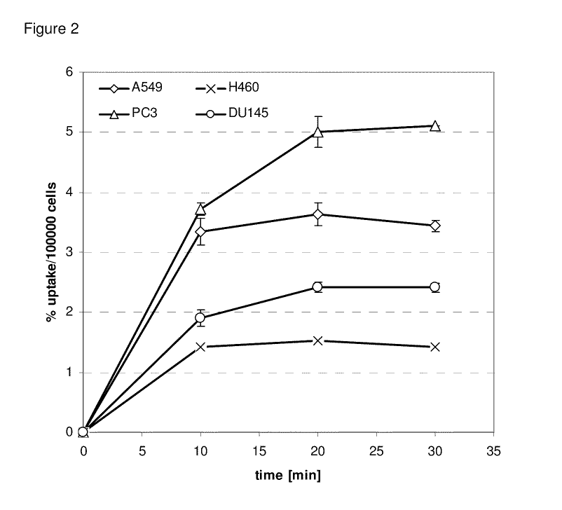

In a second experiment, the time-dependence of binding of (5R)-[¹⁸F]-fluoromethyl-L-ornithine to several tumor cell lines was determined using A549, H460 as well as PC3 (prostate) and DU145 (prostate) tumor cell lines. After 30 min incubation with 0.25 MBq up to 5% (PC3 cells) of applied dose were bound to the cells (FIG. 2).

### Example (31) (3R)-3-fluoro-L-ornithine-dihydrochloride

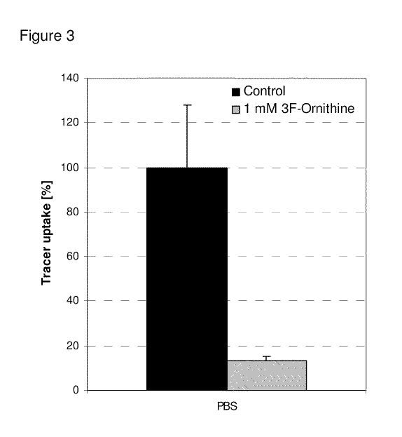

(3R)-3-fluoro-L-ornithine-dihydrochloride (31) was used in a cell competition experiment using ¹⁴C-ornithine as tracer. It was discovered, that 3-Fluoroornithine can block uptake of ¹⁴C-ornithine in A549 cells to a large extent (FIG. 3).

### Example (29) Determination of the biological activity of (4S)-[¹⁸F]-fluoro-L-ornithine in tumor cells

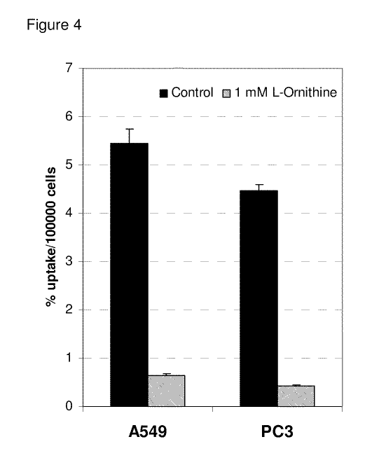

To determine the specificity of (4S)-[¹⁸F]-fluoro-L-ornithine (29), the fluorinated compound was used as tracer in a cell competition experiment in A549 as well as PC3 tumor cells against an excess of L-Ornithine (1 mM). Interestingly, it was discovered, that the uptake of (4S)-[¹⁸F]-fluoro-L-ornithine was blockable by excess of ornithine, indicating the use of the same uptake system (FIG. 4).

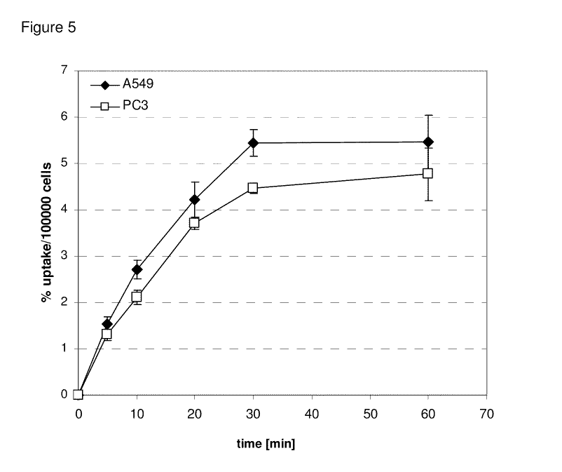

In a second experiment, A549 and PC3 cells were incubated with (4S)-[¹⁸F]-fluoro-L-ornithine for up to 60 min and the cell-bound fraction was determined. Approximately 5% of applied dose was taken up by the cells during the 60 min incubation period (FIG. 5).

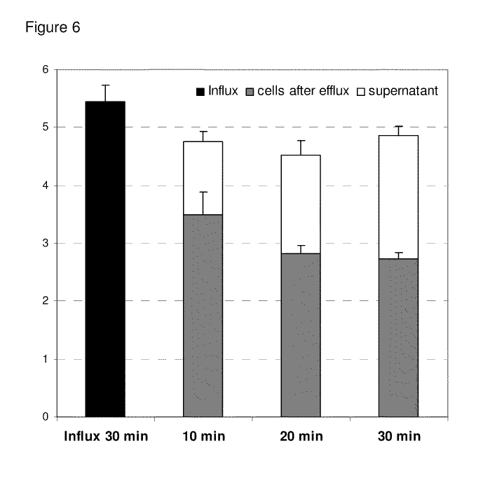

In a third experiment, the retention of activity in tumor cells was examined. A549 cells were incubated with (4S)-[¹⁸F]-fluoro-L-ornithine for 30 min. After this time, cells were incubated with new buffer (without radiotracer) for up to 30 min. The release of radioactivity into the supernatant as well as the retention inside the cells was examined. It was discovered, that approximately 66% of activity were retained in the tumor cells after 30 min under efflux conditions (FIG. 6).

Animal experiments. (4S)-[¹⁸F]-fluoro-L-ornithine was examined in NCI-H460 (human NSCLC) tumor bearing rats using PET-Imaging. PET images were obtained from 45 min after administration of the radiotracer (7.16 MBq) for 30 min. The tumor was very well visualized, with up to 2.2% injected dose per gram tumor determined by ROI analyses. Some partial defluorination was observed at later time points, resulting in uptake of the released [F18]-fluoride in the bones (FIG. 7). After the PET-imaging study (98 min p.i.) the rat was sacrificed, several organs were removed and the amount of radioactivity in tissues was measured. See Table 1. High amounts of radioactivity were observed in the tumor (1.43% ID/g), in the pancreas (2.47% ID/g) as well as in the bones (2.08% ID/g).

TABLE 1

| Organ | % ID/g |
|---|---|
| kidney | 1.18 |
| pancreas | 2.47 |
| liver | 0.39 |
| bone | 2.08 |
| brain | 0.24 |
| heart | 0.44 |
| lung | 0.44 |
| tumor | 1.43 |
| blood | 0.38 |

FIG. 1: Examination of biological activity of (5R)-[¹⁸F]-fluoromethyl-L-ornithine (38) from in a cell-competition-experiment. (NCI-H460 and A549 cells, 30 min incubation with 0.25 MBq (5R)-[¹⁸F]-fluoromethyl-L-ornithine in PBS-Puffer, concentration of L-ornithine 1 mM).

FIG. 2: Binding of (5R)-[¹⁸F]-fluoromethyl-L-ornithine (38) to several tumor cell lines. (A549, H460 (both human NSCLC) as well as PC3 and DU145 (both prostate) tumor cell lines were used and incubated with 0.25 MBq (5R)-[¹⁸F]-fluoromethyl-L-ornithine for up to 30 min. The cell-bound fraction of activity was determined after 10 min, 20 min and 30 min.

FIG. 3: The specificity of (3R)-3-fluoro-L-ornithine-dihydrochloride (31) to compete for ¹⁴C-Ornithine uptake was determined in a cell competition experiment in A549 cells. (0.1 μCi ¹⁴C-Ornithine was used as tracer, (3R)-3-fluoro-L-ornithine-dihydrochloride was used at a concentration of 1 mM, incubation period 10 min).

FIG. 4: The specificity of (4S)-[¹⁸F]-fluoro-L-ornithine (29) for uptake into tumor cells was determined in cell competition experiments using A549 as well as PC3 tumor cells. (0.25 MBq of (4S)-[¹⁸F]-fluoro-L-ornithine was used as tracer, an excess of 1 mM L-ornithine was used for saturation of uptake systems, incubation time 30 min).

FIG. 5: The time dependence of uptake of (4S)-[¹⁸F]-fluoro-L-ornithine(29) was determined. (A549 and PC3 cells were incubated with 0.25 MBq (4S)-[¹⁸F]-fluoro-L-ornithine for up to 60 min and the cell-bound fraction was determined after 10, 20, 30 and 60 min)

FIG. 6: Examination of retention of (4S)-[¹⁸F]-fluoro-L-ornithine (29) in A549 tumor cells. A549 cells were loaded with 0.25 MBq (4S)-[¹⁸F]-fluoro-L-ornithine for 30 min in PBS. After washing, the cells were incubated with new buffer (without activity) for additional 10, 20, 30 min. The release of radioactivity into the supernatant as well as the retention inside the cells was determined.

FIG. 7: PET-Imaging of (4S)-[¹⁸F]-fluoro-L-ornithine (29) in H460 tumor bearing rats. 7.16 MBq of radioactive tracer was injected i.v. into rats. PET images were obtained using the Inveon PET/CT scanner from 45 min p.i. for 30 min.

1. A compound of formula I 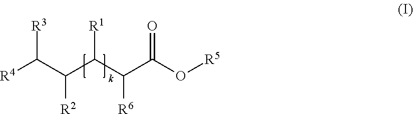
wherein
R¹, R² and R³ are selected independently and individually from the group comprising
a) hydrogen,
b) R⁷—C₁-C₁₀ alkoxy,
c) R⁷—C₁-C₁₀ alkyl,
d) R⁷—C₂-C₁₀ alkenyl,
e) R⁷—C₂-C₁₀ alkinyl,
f) (R⁷-aryl)-C₀-C₁₀alkyl,
g) (R⁷-heteroaryl)-C₀-C₁₀alkyl,
h) ((R⁷—(C₁-C₆)alkoxy)aryl)(C₀-C₁₀)alkyl),
i) R⁷,
j) hydroxyl,
k) C₆-C₁₀ aralkyl,
l) C₁-C₁₀ alkyl and
m) C₁-C₁₀ alkoxy;
R⁴ is selected from the group comprising
a) NH₂ and
b) R¹⁴;
R⁵ is selected from the group comprising
a) hydrogen,
b) Z and
c) R¹³;
R⁶ is selected from the group comprising
a) NH₂ and
b) R¹⁴;
R⁷ is selected from the group comprising
a) [¹⁹F]fluoro,
b) Chelator free radionuclide,
c) R¹⁵ and
d) R¹⁰;
R¹⁰ is selected from the group comprising R²⁰ and R³⁰;
R¹⁵ is a leaving group;
R¹⁴ is selected from the group comprising
a) N(H)(R⁹),
b) N(R⁹)₂.
c) N═C(R¹¹)(R¹²)
d) 1,3-dioxo-1,3-dihydro-2H-isoindol-2-yl (phthalimido) and
e) azido group;
R¹³ is a carboxylic acid protecting group;
R²⁰ is selected from the group comprising
a) iodo,
b) —Sn((C₁-C₆)alkyl)₃,
c)—B(OR⁶⁰)(OR⁶¹) wherein B means boron and
d)—NMe₂;
R³⁰ is hydroxyl;
Z is a metal ion equivalent;
R⁹ is an amino-protecting group;
R¹¹ and R¹² are independently and individually selected from the group comprising
a) C₁-C₅ alkyl,
b) substituted or unsubstituted aryl,
c) substituted or unsubstituted aralkyl and
d) substituted or unsubstituted heteroaryl;
R⁶⁰ and R⁶¹ are independently and individually selected from the group comprising hydrogen, (C₁-C₆)alkyl and cycloalkyl, whereas R⁶⁰ and R⁶¹ can be linked to each other by a single bond or a “methylene bridge”;
k is an integer from 1 to 4;
including all isomeric forms of said compound, including but not limited to enantiomers and diastereoisomers as well as racemic mixtures;
and any pharmaceutically acceptable salt, ester, amide, complex or prodrug thereof.

2. The compound according to claim 1 wherein
R¹, R² and R³ are selected individually and independently from the group comprising
a) hydrogen,
b) R⁷—C₁-C₆ Alkyl,
c) R⁷ and
d) C₁-C₅ Alkyl.
with the proviso that compounds of Formula I contain exactly one R⁷;

3. The compound according to claim 2 wherein
R⁷ is a chelator free radionuclide.

4. The compound according to claim 3 wherein the chelator free radionuclide is Bromo-77 [⁷⁷Br], Bromo-76 [⁷⁶Br], Oxygen-15 [¹⁵O], Nitrogen-13 [¹³N], Carbon-11 [¹¹C], iodine-123 [¹²³]iodo, iodine-124 [¹²⁴iodo], iodine-125 [¹²⁵ iodo], iodine-127 [¹²⁷iodo], iodine-131 [¹³¹iodo] or Fluorine-18 [¹⁸F], preferably Fluorine-18 [¹⁸F].

5. The compound according to claim 1 wherein Z is selected from the group comprising Na⁺, K⁺, Ca²⁺ and Mg²⁺.

6. The compound according to claim 1 wherein independently from each other
k is an integer 1 or 2, and
n is an integer 1 or 2,

7. The compound according to claim 1 selected from
(4R)—N⁵-[(benzyloxy)carbonyl]-N²-(tert-butoxycarbonyl)-4-hydroxy-L-ornithinate (26) 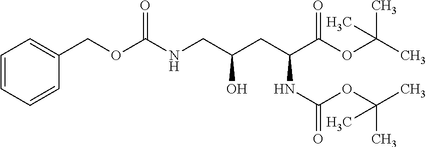
(4R)—N⁵-[(benzyloxy)carbonyl]-N²-(tert-butoxycarbonyl)-4-[(methylsulfonyl)oxy]-L-ornithinate (27): 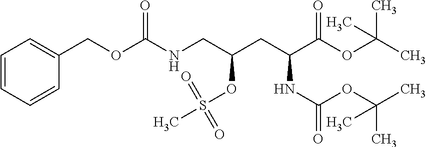
(4R)—N⁵-[(benzyloxy)carbonyl]-N²-(tert-butoxycarbonyl)-4-{[(4-methylphenyl)sulfonyl]oxy}-L-ornithinate (28): 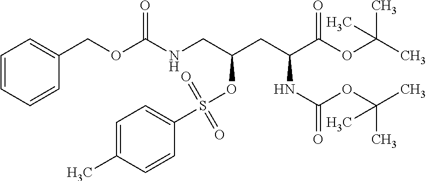
(4S)-[¹⁸F]-fluoro-L-ornithine (29) 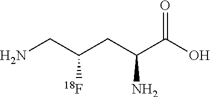
(3R)—N²,N⁵-bis(tert-butoxycarbonyl)-3-fluoro-L-ornithinate (30): 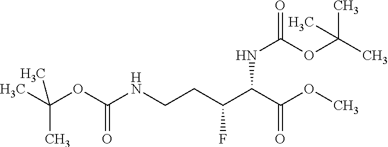
(3R)-3-Fluoro-L-ornithin (31) 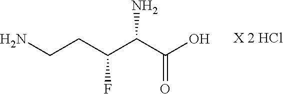
methyl-(5S)—N-(tert-butoxycarbonyl)-6-{[tert-butyl(dimethyl)silyl]oxy}-5-hydroxy-L-norleucinate (34) 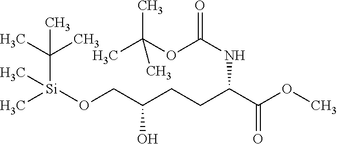
methyl-(5R)-5-azido-N-(tert-butoxycarbonyl)-6-{[tert-butyl(dimethyl)silyl]oxy}-L-norleucinate (35) 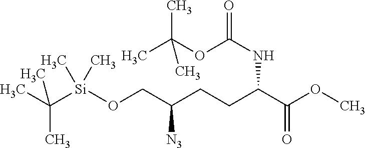
methyl-(5R)-5-azido-N-(tert-butoxycarbonyl)-6-hydroxy-L-norleucinate (36) 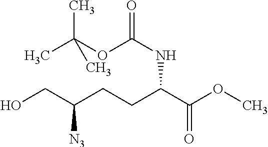
(5R)-[¹⁸F]-fluoromethyl-L-ornithine (38) 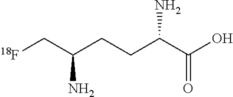
(2S)-5-amino-2-(1,3-dioxo-1,3-dihydro-2H-isoindol-2-yl)-4-fluoropentanoic acid (43) 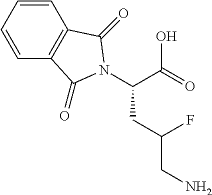
benzyl (2S)-5-azido-2-(1,3-dioxo-1,3-dihydro-2H-isoindol-2-yl)-4-fluoropentanoate (42) 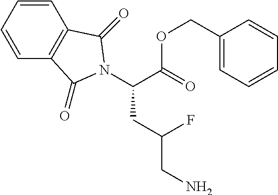 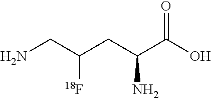
4-(¹⁸F)fluoro-L-ornithine 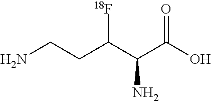
3-(¹⁸F)fluoro-L-ornithine 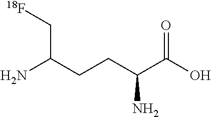
5-amino-6-(¹⁸F)fluoro-L-norleucine

8. A pharmaceutical composition comprising compounds having Formula I according to claim 1 or pharmaceutically acceptable salt of an inorganic or organic acid thereof, a hydrate, a complex, an ester, an amide, a solvate or a prodrug thereof and a pharmaceutical acceptable carrier, diluent, excipient or adjuvant.

9. A compound of Formula I according to claim 1 for use as reference compound, medicament or radiopharmaceutical.

10. A compounds of Formula I according to claim 1 for use as imaging agent.

11. The imaging agent according to claim 10 that is suitable for PET, SPECT or Micro-PET imaging or in combination with other imaging conventional method such as Computer Tomography (CT), and magnetic resonance (MR) spectroscopy of hyperproliferative diseases.

12. A method for obtaining compounds of Formula I wherein R⁷ is a chelator free radionuclide or [¹⁹F] by reacting compounds of Formula I wherein R⁷ is leaving group with a suitable labeling agent.

13. A method for obtaining compounds of Formula Ib 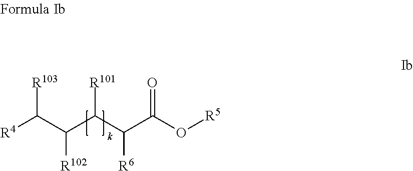
by reacting a compound of Formula V 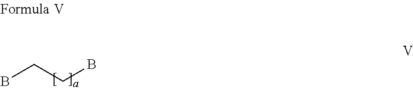
with a labeling agent comprising R⁸⁶ to yield a compound of Formula IV, 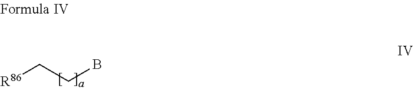
by substituting said compound of Formula IV with a compound of Formula VI 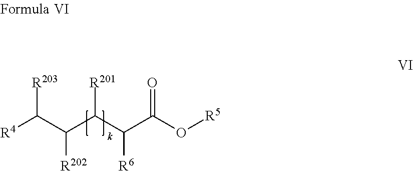
and
Optionally, deprotecting amine- and/or carboxylic-protecting group;
wherein Formula Ib is defined as bellowed
R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
d) C₆-C₁₀ aralkyl,
e) C₁-C₁₀ alkyl and
f) C₁-C₁₀ alkoxy,
with the proviso that compounds of Formula Ib comprise at least one R⁸⁶,
R⁸⁶ is a chelator free radionuclide or [¹⁹F], and
R⁴, R⁵, R⁶, and k are defined as above,
wherein Formula V is defined as bellowed
a is an integer from 0 to 5, and
B is a leaving group,
wherein Formula IV is defined as bellowed
a is an integer from 0 to 5,
B is a leaving group, and
R⁸⁶ is a chelator free radionuclide or [¹⁹F],
wherein Formula VI is defined as bellowed
R²⁰¹, R²⁰² and R²⁰³ are selected individually and independently from the group comprising
a) hydrogen,
b) ((R⁸-aryl)(C₀-C₁₀)alkyl
c) hydroxyl,
d) C₆-C₁₀ aralkyl,
e) C₁-C₁₀ alkyl and
f) C₁-C₁₀ alkoxy;
R⁸ is hydroxyl;
with the proviso that compounds of Formula VI comprise at least one R⁸,
R⁴, R⁵, R⁶, and k are defined as above.

14. A compound of Formula Ib 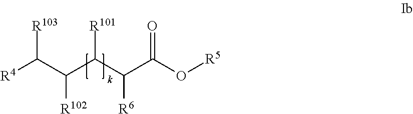
wherein
R¹⁰¹, R¹⁰² and R¹⁰³ are selected individually and independently from the group comprising
a) hydrogen,
b) ((R⁸⁶—(C₁-C₆)alkoxy)aryl)(C₀-C₁₀)alkyl)
c) hydroxyl,
d) C₆-C₁₀ aralkyl,
e) C₁-C₁₀ alkyl and
f) C₁-C₁₀ alkoxy,
with the proviso that compounds of Formula Ib comprise at least one R⁸⁶,
R⁸⁶ is a chelator free radionuclide or [¹⁹F], and
R⁴, R⁵, R⁶, and k are defined as above
including all isomeric forms of said compound, including but not limited to enantiomers and diastereoisomers as well as racemic mixtures;
and any pharmaceutically acceptable salt, ester, amide, complex or prodrug thereof.

15. A compound of Formula VI 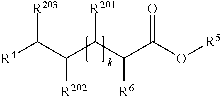
wherein
R²⁰¹, R²⁰² and R²⁰³ are selected individually and independently from the group comprising
a) hydrogen,
b) ((R⁸-aryl)(C₀-C₁₀)alkyl
c) hydroxyl,
d) C₆-C₁₀ aralkyl,
e) C₁-C₁₀ alkyl and
f) C₁-C₁₀ alkoxy;
R⁸ is hydroxyl;
with the proviso that compounds of Formula VI comprise at least one R⁸,
R⁴, R⁵, R⁶, and k are defined as above
including all isomeric forms of said compound, including but not limited to enantiomers and diastereoisomers as well as racemic mixtures;
and any pharmaceutically acceptable salt, ester, amide, complex or prodrug thereof.

16. A kit comprising a sealed vial comprising a predetermined quantity of a compound
a) compound of Formula I according to claim 1 or
b) compound of Formula V 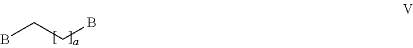
wherein B is a leaving group and a is an integer from 1-5 and a compound of Formula VI 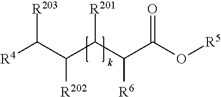
wherein
R²⁰¹, R²⁰² and R²⁰³ are selected individually and independently from the group comprising
a) hydrogen,
b) ((R⁸)(C₀-C₁₀)alkyl
c) hydroxyl,
d) C₆-C₁₀ aralkyl,
e) C₁-C₁₀ alkyl and
f) C₁-C₁₀ alkoxy;
R⁸ is hydroxyl;
with the proviso that compounds of Formula VI comprise at least one R⁸,
R⁴, R⁵, R⁶, and k are defined as above
including all isomeric forms of said compound, including but not limited to enantiomers and diastereoisomers as well as racemic mixtures;
and any pharmaceutically acceptable salt, ester, amide, complex or prodrug thereof, or a mixture of a) and b).

17. A method for obtaining compounds having Formula I wherein R¹-R⁶ are defined as above, R⁷ is R¹⁵, R⁴ is R¹⁴ and R⁵ is R¹³ as defined above,

18. A method for staging, monitoring of hyperproliferative disease progression, or monitoring response to therapy directed to hyperproliferative diseases using compound according claim 1.
